(19) United States
(12) Patent Application Publication
(10) Pub. No.: US 2010/0010088 A1
(43) Pub. Date: Jan. 14, 2010

(54) Compositions and Methods for Prevention and Treatment of Mammalian Diseases

(75) Inventors: Floyd Chilton, Winston Salem, NC (US); Fan Lu, Clemmons, NC (US)

Correspondence Address: WOMBLE CARLYLE SANDRIDGE & RICE, PLLC, ATTN: PATENT DOCKETING, P.O. BOX 7037, ATLANTA, GA, 30357-0037, US

(73) Assignee: Wake Forest University School of Medicine, Winston Salem, NC (US)

(21) Appl. No.: 12/260,134

(22) Filed: Oct. 29, 2008

Related U.S. Application Data
(60) Provisional application No. 61/001,482, filed on Nov. 1, 2007.

(51) Int. Cl. A61K 31/20; C11B 1/00; C12P 7/64; A23K 1/00; A23D 7/00; A23K 1/18; A23L 1/32; A23C 9/00; A23L 1/31; A01K 67/00

(52) U.S. Cl. 514/560; 554/8; 435/134; 426/7; 426/601; 426/2; 426/614; 426/580; 426/641; 800/8

(57) ABSTRACT

The present invention discloses processes of making a polyunsaturated fatty acid compositions, and compositions thereof. Thus, one method of making a polyunsaturated fatty acid compositions comprises at least 8% polyunsaturated fatty acids, the process comprising extracting the fatty acids from a microalgae, wherein the fatty acids can be (a) GLA in an amount of 1% to 10% of total fatty acids; (b) SDA in an amount of 5% to 50% of total fatty acids; (c) EPA in an amount of 2% to 30% of total fatty acids, and (d) DHA in an amount of 2% to 30% of total fatty acids, wherein a polyunsaturated fatty acid composition is produced comprising at least 8% polyunsaturated fatty acids. Additional processes of making polyunsaturated fatty acid compositions, animal feed additives, and animal products are disclosed and the compositions, feed additives and products thereof.

### FIELD OF THE INVENTION

This invention relates generally to the fields of lipid metabolism and dietary supplementation. More particularly, it concerns compositions and methods for preventing and treating mammalian diseases using combinations of polyunsaturated fatty acids from different species of microalgae.

### BACKGROUND OF THE INVENTION

Omega-3 fatty acids are essential for normal human growth and development, and their therapeutic and preventative benefits with regard to cardiovascular disease and rheumatoid arthritis have been well documented (James et al., A. J. Clin. Nutr. 77: 1140-1145 (2003); Simopoulos, A. J. Clin, Nutr. 70: 560S-569S (1999)). Multiple studies have documented a protective role of fish oil and n-3 polyunsaturated fatty acids (PUFAs) with regard to the development of cardiovascular diseases. The cardioprotective benefits of fish oil have been largely attributed to 20 and 22 carbon fatty acids such as eicosapentanoic acid (EPA, 20:5n-3) and docosahexanoic acid (DHA, 22:6, n-3) whose enrichment in cells and plasma lipoproteins results in decreased inflammation, thrombosis, blood pressure, arrhythmias, endothelial activation, and plasma triglyceride (TG) concentrations.

Mammals, including humans, can synthesize saturated fatty acids and monounsaturated (n-9) fatty acids but cannot synthesize either the (n-6) or the (n-3) double bond. The (n-3) and (n-6) fatty acids are essential components in cell membrane phospholipids and as a substrate for various enzymes; thus fatty acids containing these bonds are essential fatty acids and must be obtained in the diet. The (n-6) fatty acids are consumed primarily as linoleic acid [18:2(n-6)] from vegetable oils and arachidonic acid [AA, 20:4(n-6)] from meats. The (n-3) fatty acids may be consumed as y-linolenic acid [18:3(n-3)] from some vegetable oils. Longer-chain (n-3) fatty acids, mainly EPA and docosahexaenoic acid [DHA, 22:6(n-3)], are found in fish and fish oils (Hardman, J. Nutr. 134: 3427S-3430S (2004)).

In spite of the overwhelming evidence for the beneficial effects of fish oil, the consumption of n-3 PUFAs in the North American population is very low. Since the (n-3)-and (n-6) fatty acids cannot be interconverted in humans, the balance between (n-3) and (n-6) fatty acids in humans can only be achieved through appropriate diets. However, the current Western diet contains predominantly (n-6) fatty acids with a small portion of (n-3) fatty acids. In fact, it is estimated that actual dietary intakes of fatty acid from fish oil are as low as one-tenth of the levels recommended by the American Heart Association (Ursin, J. Nutr. 133: 4271-4272 (2003)). Such an imbalance in (n-3) and (n-6) fatty acids has been linked to various diseases, including asthma, cardiovascular diseases, arthritis, cancer.

Research has revealed that (n-3) and (n-6) fatty acids affect the various disease conditions through the action of two types of enzymes: cyclooxygenase (COX) and lipoxygenase (LOX). COX and LOX act on 20-carbon fatty acids to produce cell-signaling molecules. COX activity on AA or EPA produces prostaglandins or thromboxanes; LOX activity on AA or EPA produces the leukotrienes. The 2-series prostaglandins produced from AA tend to be proinflammatory and proproliferative in most tissues. The 3-series prostaglandins produced from EPA tend to be less promotional for inflammation and proliferation. Thus, EPA-derived prostaglandins are less favorable for inflammation and for the development and the growth of cancer cells (Hardman, J. Nutr. 134: 3427S-3430S (2004)).

An alternative approach to affecting inflammatory diseases has been to supplement diets with the 18-carbon polyunsaturated fatty acid of the (n-6) series, y-linolenic acid (GLA, 18:3, n-3). This fatty acid is found primarily in the oils of the evening primrose and borage plants and to a lesser extent in meats and eggs. Animal data as well as some clinical studies suggest that dietary supplementation with GLA may attenuate the signs and symptoms of 20 chronic inflammatory diseases including rheumatoid arthritis and atopic deimatitis. Echium oil, another botanical oil, which contains stearidonic acid (SDA, 18:4, n-3), has been shown to have protective effects in hypertriglyceridemic patients.

However, a major concern in many dietary studies to date is that various sources of the PUFAs, whether it be fish oil, borage, evening primrose or echium oil or combinations of these oils, provide active ingredients (certain PUFAs) that are anti-inflammatory, but they also provide n-6 fatty acids that are potentially pro-inflammatory or that block the anti-inflammatory effects of the active PUFAs. Two such fatty acids are AA and linoleic acid [18:2(n-6)]. The n-6 fatty acids are consumed primarily as linoleic acid from vegetable oils and AA from meats. Linoleic acid is converted to AA by a series of desaturation and elongation steps. The high amount of dietary linoleic acid is the primary culprit that has resulted in the major imbalance in omega 6 to omega 3 fatty acids observed in western nations. Diets high in linoleic acid have been demonstrated to be pro-inflammatory in several animal models.

Arachidonic acid is a twenty carbon n-6 fatty acid that is converted in mammals to products called leukotrienes, prostaglandins and thromboxanes. These products induce inflammation, and blocking their production utilizing drugs such as aspirin, ibuprophen, celecoxib (Celebrex™), and montelukast sodium (Singulair™) reduces signs and symptoms of inflammatory diseases including asthma and arthritis. In addition to the importance of AA in producing pro-inflammatory products, AA also regulates gene expression in mammals through transcription factors such as peroxisome proliferator-activated receptors (PPAR)-alpha leading to low level whole body inflammation. As indicated above, recent studies reveal that AA is present in high concentrations in many items in our food supply. Ironically, it is found in high concentrations in certain fish. AA in human diets has been correlated with increased levels of pro-inflammatory products, platelet aggregation and atherosclerosis.

### SUMMARY OF THE INVENTION

A major advance in the design and development of formulations containing anti-inflammatory fatty acids would be to develop complex oils that contain optimal ratios of anti-inflammatory or anti-cardiovascular disease fatty acids in which non-beneficial or harmful fatty acids are minimized. This may allow for an increase in the dietary intake of anti-inflammatory or anti-cardiovascular disease fatty acids and, thus, allow management and treatment of certain preventable diseases and promote human well-being.

Accordingly, the present invention is directed to processes of making anti-inflammatory fatty acid compositions derived from microalgae. The invention is further directed to the compositions and methods of using the compositions.

In an embodiment, a process of making a polyunsaturated fatty acid composition comprising at least 8% polyunsaturated fatty acids is disclosed; the process comprising: extracting the polyunsaturated fatty acids from a microalgae, wherein (a) GLA is in an amount of 1% to 10% of total fatty acids; (b) SDA is in an amount of 5% to 50% of total fatty acids; (c) EPA is in an amount of 2% to 30% of total fatty acids; and (d) DHA is in an amount of 2% to 30% of total fatty acids; wherein composition comprises at least 8% polyunsaturated fatty acids.

In another embodiment, a process of making a composition comprising at least 5% stearidonic acid is disclosed, the process-comprising: (a) cultivating a microalgae to produce a microalgal biomass; and either (b) extracting said microalgal oil from said microalgal biomass; or (c) removing water from said microalgal biomass to achieve a solids content from about 5 to 100%; wherein the composition comprises at least 5% stearidonic acid.

In yet another embodiment, a process of making an animal feed additive comprising fatty acids from a microalgae is disclosed, the process comprising: (a) cultivating microalgae to produce a microalgal biomass; and either (b) extracting microalgae oil from said microalgal biomass to produce a microalgal oil; or (c) removing water from said microalgal biomass to produce a microalgal biomass with a solids content from about 5% to 100%; wherein the animal feed additive comprises fatty acids from a microalgae.

In a further embodiment, a process of making an animal feed additive comprising at least 8% polyunsaturated fatty acids is disclosed; the process comprising: extracting the fatty acids from a microalgae, wherein (a) GLA is in an amount of 1% to 10% of total fatty acids; (b) SDA is in an amount of 5% to 50% of total fatty acids; (c) EPA is in an amount of 2% to 30% of total fatty acids; and (d) DHA is in an amount of 2% to 30% of total fatty acids, wherein the animal feed additive comprises at least 8% polyunsaturated fatty acids.

In yet a further embodiment, a process of producing an animal having an increased tissue content of long chain omega-3 fatty acids, the method comprising feeding to an animal an animal feed additive comprising fatty acids collected from microalgae, the animal feed additive further comprising: (a) a microalgal oil extracted from a cultivated microalgae biomass and/or (b) a microalgal biomass from a cultivated microalgae, wherein water is removed from microalgal biomass to achieve a solids content from about 5 to 100%; wherein an animal is produced having increased tissue content of long chain omega-3 fatty acids.

In an additional embodiment, a process of producing an animal having an increased tissue content of long chain omega-3 fatty acids is provided, the process comprising feeding to an animal an animal feed additive comprising at least 8% polyunsaturated fatty acids; the animal feed additive comprising fatty acids extracted from a microalgae, wherein (a) GLA is in an amount of 1% to 10% of total fatty acids; (b) SDA is in an amount of 5% to 50% of total fatty acids; (c) EPA is in an amount of 2% to 30% of total fatty acids; and (d) DHA is in an amount of 2% to 30% of total fatty acids; wherein an animal is produced having an increased tissue content of long chain omega-3 fatty acids.

In a subsequent embodiment, a method of treating a mammalian disease in a subject in need thereof by administration to the subject a therapeutically effective amount of a polyunsaturated fatty acid composition comprising at least 8% polyunsaturated fatty acids is provided; the composition further comprising fatty acids extracted from a microalgae, wherein the microalgae fatty acid extract comprises: (a) GLA in an amount of 1% to 10% of total fatty acids; (b) SDA in an amount of 5% to 50% of total fatty acids; (c) EPA in an amount of 2% to 30% of total fatty acids, and (d) DHA in an amount of 2% to 30% of total fatty acids.

In another subsequent embodiment, a method of treating a mammalian disease in a subject in need thereof by administration to the subject a therapeutically effective amount of a composition comprising at least 5% SDA is provided, the composition comprising either (a) a microalgal oil extracted from a cultivated microalgae biomass or (b) a microalgal biomass from a cultivated microalgae, wherein water is removed from microalgal biomass to achieve a solids content from about 5 to 100%.

In a further subsequent embodiment, a polyunsaturated fatty acid composition comprising at least 8% polyunsaturated fatty acids is provided; the composition comprising fatty acids extracted from a microalgae, wherein the microalgae fatty acid extract comprises: (a) GLA in an amount of 1% to 10% of total fatty acids; (b) SDA in an amount of 5% to 50% of total fatty acids; (c) EPA in an amount of 2% to 30% of total fatty acids; and (d) DHA in an amount of 2% to 30% of total fatty acids.

In yet a further subsequent embodiment, a composition comprising at least 5% SDA is provided, the composition comprising either: (a) microalgal oil extracted from a cultivated microalgae biomass or (b) a microalgal biomass from a cultivated microalgae, wherein water is removed from microalgal biomass to achieve a solids content from about 5 to 100%.

In an additional subsequent embodiment, a food product is provided comprising: (a) from 0.01-99.99 percent by weight of a composition comprising at least 8% polyunsaturated fatty acids, wherein the fatty acids are extracted from a microalgae, further wherein the microalgal fatty acid extract comprises: (i) GLA in an amount of 1% to 10% of total fatty acids; (ii) SDA in an amount of 5% to 50% of total fatty acids; (iii) EPA in an amount of 2% to 30% of total fatty acids, and (iv) DHA in an amount of 2% to 30% of total fatty acids; in combination with (b) from 99.99-0.01 percent by weight of at least one additional ingredient selected from the group consisting of proteins, carbohydrates and fiber, and combinations thereof.

Further embodiments of the invention provide a food product comprising: (a) from 0.01-99.99 percent by weight of a composition comprising at least 5% stearidonic acid, the composition comprising either: (i) a microalgal oil extracted from a cultivated microalgae biomass or (ii) a microalgal biomass from a cultivated microalgae, wherein water is removed from microalgal biomass to achieve a solids content from about 5 to 100% weight percent; in combination with (b) from 99.99 to 0.01 percent by weight of at least one additional ingredient selected from the group consisting of proteins, carbohydrates and fiber, and combinations thereof.

In other embodiments of the invention, an animal feed additive is provided wherein the animal feed additive comprises fatty acids collected from microalgae either in the form of: a) a microalgal oil extracted from a cultivated microalgae biomass or (b) a microalgal biomass from a cultivated microalgae, wherein water is removed from microalgal biomass to achieve a solids content from about 5 to 100% weight percent.

Additionally provided herein is an animal feed additive comprising at least 8% polyunsaturated fatty acids; the additive comprising fatty acids extracted from a microalgae, wherein the microalgal fatty acid extract further comprises: (a) GLA in an amount of 1% to 10% of total fatty acids; (b) SDA in an amount of 5% to 50% of total fatty acids; (c) EPA in an amount of 2% to 30% of total fatty acids; and (d) DHA in an amount of 2% to 30% of total fatty acids.

An other embodiment of the invention includes an animal product produced by feeding to an animal an animal feed additive comprising fatty acids collected from microalgae either in the form of: (a) a microalgal oil extracted from a cultivated microalgae biomass or (b) a microalgal biomass from a cultivated microalgae, wherein water is removed from microalgal biomass to achieve a solids content from about 5 to 100% weight percent.

Still other embodiments of the invention provide an animal product produced by feeding to an animal an animal feed additive comprising at least 8% polyunsaturated fatty acids; the additive comprising fatty acids extracted from a microalgae, wherein the microalgal fatty acid extract further comprises (a) GLA in an amount of 1% to 10% of total fatty acids; (b) SDA in an amount of 5% to 50% of total fatty acids; (c) EPA in an amount of 2% to 30% of total fatty acids, and (d) DHA in an amount of 2% to 30% of total fatty acids.

The foregoing and other aspects of the present invention will now be described in more detail with respect to other embodiments described herein. It should be appreciated that the invention can be embodied in different forms and should not be construed as limited to the embodiments set forth herein. Rather, these embodiments are provided so that this disclosure will be thorough and complete, and will fully convey the scope of the invention to those skilled in the art.

### BRIEF DESCRIPTION OF THE FIGURES

FIG. 1 shows the fatty acid profiles of Rhodomonas salina and Amphidinium carterae.

FIG. 2 shows the effect of light intensity on chlorophyll-a concentration (A) and cell number (B) in Rhodomonas salina.

FIG. 3 shows the effect of temperature on chlorophyll-a concentration (A) and cell number (B) in Rhodomonas salina.

FIG. 4 shows the effect of light intensity (A) and temperature (B) on total pigment profile in Rhodomonas salina.

FIG. 5 shows the effect of light intensity on chlorophyll-a concentration (A) and cell number (B) in Amphidinium. carterae.

FIG. 6 shows the effect of temperature on chlorophyll-a concentration (A) and cell number (B) in Amphidinium carterae.

FIG. 7 shows the effects of light intensity (A) and temperature (B) on total pigment profile in Amphidinium carterae.

FIG. 8 presents the results of the cytotoxicity tests of Rhodomonas salina and Amphidinium carterae.

FIG. 9 shows the effects of culture stage and nutrition on fatty acid accumulation in Rhodomonas salina grown at 28° C.

FIG. 10 shows the effect of temperature on SDA content Rhodomonas salina and Amphidinium carterae.

FIG. 11 shows the effects of light intensity on SDA content in Rhodomonas salina.

### DETAILED DESCRIPTION

As used herein, the phrase “therapeutically effective amount” refers to an amount of a compound or composition that is sufficient to produce the desired effect, which can be a therapeutic or agricultural effect. The therapeutically effective amount will vary with the application for which the compound or composition is being employed, the microorganism and/or the age and physical condition of the subject, the severity of the condition, the duration of the treatment, the nature of any concurrent treatment, the pharmaceutically or agriculturally acceptable carrier used, and like factors within the knowledge and expertise of those skilled in the art. An appropriate “therapeutically effective amount” in any individual case can be determined by one of ordinary skill in the art by reference to the pertinent texts and literature and/or by using routine experimentation. (See, for example for pharmaceutical applications, Remington, The Science And Practice of Pharmacy (9th Ed. 1995).

Disclosed is a novel process for producing polyunsaturated fatty acids, and a novel composition of polyunsaturated fatty acids derived from microalgae.

Generally, the process of making a polyunsaturated fatty acid composition comprising at least 8% polyunsaturated fatty acids comprises: extracting at least one fatty acid from a microalgae, wherein (a) GLA is in an amount of 1% to 10% of total fatty acids; (b) SDA is in an amount of 5% to 50% of total fatty acids; (c) EPA is in an amount of 2% to 30% of total fatty acids, and (d) DHA is in an amount of 2% to 30% of total fatty acids, wherein the composition comprises at least 8% polyunsaturated fatty acids.

In one embodiment, the microalgae can be a mixture of different microalgal species. In some embodiments, one of the fatty acids, GLA, SDA, EPA or DHA, is not included in the composition. In other aspects of the invention the polyunsaturated fatty acid composition is supplemented with polyunsaturated fatty acids from other sources including, but not limited to plant sources. Plant sources of polyunsaturated fatty acids include, but are not limited to, borage, black currant, echium and primrose.

In some embodiments, the polyunsaturated fatty acid composition produced by the process of the invention can comprise polyunsaturated fatty acids at a concentration in a range from 5% to 35%. Thus, the polyunsaturated fatty acid composition can comprise polyunsaturated fatty acids at a concentration of 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, and the like. In other embodiments, the polyunsaturated fatty acid composition can comprise polyunsaturated fatty acids in a range from 5% to 7%, 5% to 8%, 5% to 10%, 5% to 12%, 5% to 15%, 5% to 20%, 5% to 25%, 5% to 30%, 6% to 8%, 6% to 10%, 6% to 12%, 6% to 15%, 6% to 20%, 6% to 25%, 6% to 35%, 7% to 9%, 7% to 11%, 7% to 13%, 7% to 14%, 7% to 15%, 7% to 20%, 7% to 25%, 7% to 30%, 7% to 35%, 8% to 10%, 8% to 12%, 8% to 14%, 8% to 15%, 8% to 20%, 8% to 25%, 8% to 35%, 9% to 11%, 9% to 13%, 9% to 15%, 9% to 20%, 9% to 25%, 9% to 30%, 9% to 35%, 10% to 12%, 10% to 13%, 10% to 14%, 10% to 15%, 10% to 20%, 10% to 25%, 10% to 30%, 10% to 35%, 15% to 20%, 15% to 25%, 15% to 30%, 20% to 25%, 20% to 30%, 20% to 35%, 25% to 30%, 25% to 35%, 30% to 35%, and the like. In one embodiment, the polyunsaturated fatty acid composition comprises polyunsaturated fatty acids at a concentration of at least 8%.

In other embodiments, the amount of GLA that can be included in the composition is in a range from 1% to 10% of total fatty acids. Thus, the GLA can be included in the composition in an amount of total fatty acids of 1%, 2%, 3%, 4%, 5%, 6%, 7%, 8%, 9%, 10%, and the like. In other embodiments, the GLA can be included in the composition in an amount of total fatty acids in a range from 1 % to 3%, 1 % to 5%, 1% to 7%, 1% to 9%, 2% to 4%, 2% to 6%, 2% to 8%, 2% to 10%, 3% to 5%, 3% to 7%, 3% to 9%, 3% to 10%, 4% to 6%, 4% to 8%, 4% to 10%, 5% to 7%, 5% to 8%, 5% to 9%, 5% to 10%, 6% to 8%, 6% to 9%, 6% to 10%, 7% to 9%, 7% to 10%, 8% to 10%, 9% to 10%, and the like.

In some embodiments, the amount of SDA that is included in the composition of the present invention is in a range from 5% to 50% of total fatty acids. Thus, the SDA can be provided in the composition in an amount of total fatty acids of 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, and the like. In other embodiments, the SDA can be included in the composition in an amount of total fatty acids in a range from 5% to 10%, 5% to 15%, 5% to 20%, 5% to 25%, 5% to 30%, 5% to 35%, 5% to 40%, 5% to 45%, 5% to 50%, 10% to 15%, 10% to 20%, 10% to 25%, 10% to 30%, 10% to 35%, 10% to 40%, 5 10% to 45%, 10% to 50%, 20% to 25%, 20% to 30%, 20% to 35%, 20% to 40%, 20% to 45%, 20% to 50%, 25% to 30%, 25% to 35%, 25% to 40%, 25% to 45%, 25% to 50%, 30% to 35%, 30% to 40%, 30% to 45%, 30% to 50%, 35% to 40%, 35% to 45%, 35% to 50%, 40% to 45%, 40% to 50%, 45% to 50%, and the like.

In other embodiments, the EPA can be included in the composition in a range from 10 2% to 30% of total fatty acids. Thus, the EPA can be provided in the composition in an amount of total fatty acids of 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, and the like. In other embodiments, the EPA can be included in the composition in an amount of percent of total fatty acids in a range from 1% to 5%, 1% to 10%, 1% to 15%, 1% 15 to 20%, 1% to 25%, 1% to 30%, 5% to 10%, 5% to 15%, 5% to 20%, 5% to 25%, 5% to 30%, 10% to 15%, 10% to 20%, 10% to 25%, 10% to 30%, 15% to 20%, 15% to 25%, 15% to 30%, 20% to 25%, 20% to 30%, 25% to 30%, and the like.

In some embodiments of the present invention, the DHA can be included in the composition in a range from 2% to 30% of total fatty acids. Thus, the DHA can be provided 20 in the composition in an amount of total fatty acids of 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, and the like. In other embodiments, the DHA can be included in the composition in an amount of total fatty acids in a range from 1% to 5%, 1% to 10%, 1% to 15%, 1% to 20%, 1% to 25%, 1% to 30%, 5% to 10%, 5% to 15%, 5% to 20%, 5% to 25%, 5% to 30%, 10% to 15%, 10% to 20%, 10% to 25%, 10% to 30%, 15% to 20%, 15% to 25%, 15% to 30%, 20% to 25%, 20% to 30%, 25% to 30%, and the like.

Other aspects of the invention provide a process of making a composition comprising at least 5% SDA, the process comprising: (a) cultivating a microalgae to produce a microalgal biomass; and either (b) extracting said microalgal oil from said microalgal biomass; or (c) removing water from said microalgal biomass to achieve a solids content from about 5 to 100% weight percent; wherein a composition is produced comprising at least 5% stearidonic acid.

In some embodiments, the SDA is in a triglyceride form. In other embodiments, the SDA is not in a phospholipid form.

In some embodiments, the SDA is present in the composition in an amount in a range from 2% to 10%. Thus, the SDA is present in the composition in an amount of 2%, 3%, 4%, 5%, 6%, 7%, 8%, 9%, 10%, and the like. In other embodiments, the SDA can be included in the composition in a range from 2% to 4%, 2% to 6%, 2% to 8%, 2% to 10%, 3% to 5%, 3% to 7%, 3% to 9%, 3% to 10%, 4% to 6%, 4% to 8%, 4% to 10%, 5% to 7%, 5% to 8%, 5% to 9%, 5% to 10%, 6% to 8%, 6% to 9%, 6% to 10%, 7% to 9%, 7% to 10%, 8% to 10%, 9% to 10%, and the like.

Additional embodiments of the invention include processes of making animal feed additives. Thus, one aspect of the present invention is a process of making an animal feed additive comprising polyunsaturated fatty acids from a microalgae, the process comprising: (a) cultivating microalgae to produce a microalagal biomass; and either (b) extracting microalgae oil from said microalgal biomass to produce a microalgal oil; or (c) removing water from said microalgal biomass to produce a microalgal biomass with a solids content from about 5% to 100% weight percent; wherein the animal feed additive comprises poluyunsaturated fatty acids from microalgae.

In some embodiments, the fatty acids collected from the microalgae are short chain omega-3 fatty acids. Short chain omega-3 fatty acids include but are not limited to SDA and alpha linolenic acid (ALA).

In further embodiments, the microalgal oil extracted from the microalgal biomass can be combined with a microalgal biomass with a solids content from about 5% to 100% weight percent.

An additional aspect of the invention provides a process of making an animal feed additive comprising at least 8% polyunsaturated fatty acids; the process comprising: extracting the fatty acids from a microalgae, wherein the fatty acids may include (a) GLA is in an amount of 1% to 10% of total fatty acids; (b) SDA is in an amount of 5% to 50% of total fatty acids; (c) EPA is in an amount of 2% to 30% of total fatty acids; and (d) DHA is in an amount of 2% to 30% of total fatty acids; wherein the animal feed additive comprises at least 8% polyunsaturated fatty acids.

In some embodiments, the animal feed additive produced by the process of the invention can comprise polyunsaturated fatty acids at a concentration in a range of 5% to 35%. Thus, the animal feed additive can comprise polyunsaturated fatty acids at a concentration of 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, and the like. In other embodiments, the animal feed additive can comprise polyunsaturated fatty acids at a concentration in a range from 5% to 7%, 5% to 8%, 5% to 10%, 5% to 12%, 5% to 15%, 5% to 20%, 5% to 25%, 5% to 30%, 6% to 8%, 6% to 10%, 6% to 12%, 6% to 15%, 6% to 20%, 6% to 25%, 6% to 35%, 7% to 9%, 7% to 11%, 7% to 13%, 7% to 14%, 7% to 15%, 7% to 20%, 7% to 25%, 7% to 30%, 7% to 35%, 8% to 10%, 8% to 12%, 8% to 14%, 8% to 15%, 8% to 20%, 8%, to 25%, 8% to 35%, 9% to 11%, 9% to 13%, 9% to 15%, 9% to 20%, 9% to 25%, 9% to 30%, 9% to 35%, 10% to 12%, 10% to 13%, 10% to 14%, 10% to 15%, 10% to 20%, 10% to 25%, 10% to 30%, 10% to 35%, 15% to 20%, 15% to 25%, 15% to 30%, 20% to 25%, 20% to 30%, 20% to 35%, 25% to 30%, 25% to 35%, 30% to 35%, and the like. In one embodiment, the animal feed additive comprises polyunsaturated fatty acids at a concentration of at least 8%.

In further embodiments, the amount of GLA that can be included in the animal feed additive is in a range from 1% to 10% of total fatty acids. Thus, the GLA can be included in the animal feed additive in an amount of total fatty acids of 1%, 2%, 3%, 4%, 5%, 6%, 7%, 8%, 9%, 10%, and the like. In other embodiments, the GLA can be included in the animal feed additive in an amount of total fatty acids in a range from 1% to 3%, 1% to 5%, 1% to 7%, 1% to 9%, 2% to 4%, 2% to 6%, 2% to 8%, 2% to 10%, 3% to 5%, 3% to 7%, 3% to 9%, 3% to 10%, 4% to 6%, 4% to 8%, 4% to 10%, 5% to 7%, 5% to 8%, 5% to 9%, 5% to 10%, 6% to 8%, 6% to 9%, 6% to 10%, 7% to 9%, 7% to 10%, 8% to 10%, 9% to 10%, and the like.

In still further embodiments, the amount of SDA that is included in the animal feed additive of the present invention is in a range from 5% to 50% of total fatty acids. Thus, the animal feed additive can comprise SDA in an amount of total fatty acids of 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, and the like. In other embodiments, the SDA can be included in the animal feed additive in an amount of total fatty acids in a range from 5% to 10%, 5% to 15%, 5% to 20%, 5% to 25%, 5% to 30%, 5% to 35%, 5% to 40%, 5% to 45%, 5% to 50%, 10% to 15%, 10% to 20%, 10% to 25%, 10% to 30%, 10% to 35%, 10% to 40%, 10% to 45%, 10% to 50%, 20% to 25%, 20% to 30%, 20% to 35%, 20% to 40%, 20% to 45%, 20%. to 50%, 25% to 30%, 25% to 35%, 25% to 40%, 25% to 45%, 25% to 50%, 30% to 35%, 30% to 40%, 30% to 45%, 30% to 50%, 35% to 40%, 35% to 45%, 35% to 50%, 40% to 45%, 40% to 50%, 45% to 50%, and the like.

In other embodiments, the EPA can be included in the animal feed additive in a range from 2% to 30% of total fatty acids. Thus, the EPA can be provided in the animal feed additive in an amount of total fatty acids of 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, and the like. In other embodiments, the EPA can be included in the animal feed additive in an amount of percent of total fatty acids in a range from 1% to 5%, 1% to 10%, 1% to 15%, 1% to 20%, 1% to 25%, 1% to 30%, 5% to 10%, 5% to 15%, 5% to 20%, 5% to 25%, 5% to 30%, 10% to 15%, 10% to 20%, 10% to 25%, 10% to 30%, 15% to 20%, 15% to 25%, 15% to 30%, 20% to 25%, 20% to 30%, 25% to 30%, and the like.

In some embodiments of the present invention, the DHA can be included of the animal feed additive in a range from 2% to 30% of total fatty acids. Thus, the DHA can be provided in the animal feed additive in an amount of total fatty acids of 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, and the like. In other embodiments, the DHA can be included in the animal feed additive in an amount of total fatty acids in a range from 1% to 5%, 1% to 10%, 1% to 15%, 1% to 20%, 1% to 25%, 1% to 30%,5% to 10%, 5% to 15%, 5% to 20%, 5% to 25%, 5% to 30%, 10% to 15%, 10% to 20%, 10% to 25%, 10% to 30%, 15% to 20%, 15% to 25%, 15% to 30%, 20% to 25%, 20% to 30%, 25% to 30%, and the like.

Further embodiments of the present invention provide a process of making an animal feed additive comprising at least 5% SDA, the process comprising: (a) cultivating a microalgae to produce a microalgal biomass; and either (b) extracting said microalgal oil from said microalgal biomass; or (c) removing water from said microalgal biomass to achieve a solids content from about 5 to 100% weight percent; wherein an animal feed additive is produced comprising at least 5% SDA.

In some embodiments, the SDA produced by the process of the invention is present in the composition in an amount in a range from 2% to 10%. Thus, the SDA is present in the composition in an amount of 2%, 3%, 4%, 5%, 6%, 7%, 8%, 9%, 10%, and the like. In other embodiments, the SDA can be included in the composition in a range from 2% to 4%, 2% to 6%, 2% to 8%, 2% to 10%, 3% to 5%, 3% to 7%, 3% to 9%, 3% to 10%, 4% to 6%, 4% to 8%, 4% to 10%, 5% to 7%, 5% to 8%, 5% to 9%, 5% to 10%, 6% to 8%, 6% to 9%, 6% to 10%, 7% to 9%, 7% to 10%, 8% to 10%, 9% to 10%, and the like.

A feed additive according to the present invention can be combined with other food components to produce processed animal feed products. Such other food components include one or more enzyme supplements, vitamin food additives and mineral food additives. The resulting (combined) feed additive, including possibly several different types of compounds can then be mixed in an appropriate amount with the other food components such as cereal and plant proteins to form a processed food product. Processing of these components into a processed food product can be performed using any of the currently used processing apparatuses. The animal feed additives of the present invention may be used as a supplement in animal feed by itself, in addition with vitamins, minerals, other feed enzymes, agricultural co-products (e.g., wheat middlings or corn gluten meal), or in a combination therewith.

Additional embodiments of the invention provide processes of producing an animal having increased tissue content of long chain omega-3 fatty acids, the process comprising feeding to an animal an animal feed additive described herein. The increase in tissue content of long chain omega-3 fatty acids is relative to that of an animal not fed the animal feed additives of the present invention.

Thus, one aspect of the present invention provides a process of producing an animal having an increased tissue content of long chain omega-3 fatty acids, the process comprising feeding to an animal an animal feed additive comprising fatty acids collected from microalgae, the animal feed additive further comprising: (a) a microalgal oil extracted from a cultivated microalgae biomass and/or (b) a microalgal biomass from a cultivated microalgae, wherein water is removed from microalgal biomass to achieve a solids content from about 5 to 100% weight percent, wherein an animal is produced having an increased tissue content of long chain omega-3 fatty acids.

In some embodiments, a process of producing an animal having an increased tissue content of long chain omega-3 fatty acids is provided, the process comprising feeding to an animal an animal feed additive comprising at least 8% polyunsaturated fatty acids; the animal feed additive comprising fatty acids extracted from a microalgae, wherein the fatty acids can be (a) GLA in an amount of 1% to 10% of total fatty acids; (b) SDA in an amount of 5% to 50% of total fatty acids; (c) EPA in an amount of 2% to 30% of total fatty acids; and (d) DHA in an amount of 2% to 30% of total fatty acids; wherein an animal is produced having an increased tissue content of long chain omega-3 fatty acids.

In other embodiments, a process of producing an animal having an increased tissue content of long chain omega-3 fatty acids is provided, the process comprising feeding to an animal an animal feed additive comprising at least 5% SDA, the animal feed additive comprising either (a) a microalgal oil extracted from a cultivated microalgae biomass or (b) a microalgal biomass from a cultivated microalgae, wherein water is removed from microalgal biomass of (b) to achieve a solids content from about 5 to 100% weight percent.

An animal of the present invention includes, but is not limited to, any animal whose eggs, meat, milk or other products are consumed by humans or other animals. Thus, animals of the invention include, but are not limited to, fish, poultry (chickens, turkeys, ducks, etc.), pigs, sheep, goats, rabbits, beef and dairy cattle. The term “tissue content” as used herein refers to the various parts of the animal body, including but not limited to muscle, bone, skin, hair, and blood.

The present invention additionally provides methods for treating a mammalian disease in a subject in need thereof by administration to said subject a therapeutically effective amount of the compositions of the present invention. In some embodiments, the mammalian diseases that are treated include, but are not limited to, cardiovascular diseases, inflammatory diseases, and various cancer diseases. In other embodiments, the cardiovascular diseases to be treated include, but are not limited to, hypertriglyceridemia, coronary heart disease, stroke, acute myocardial infarction and atherosclerosis. In further embodiments, the inflammatory diseases to be treated include, but are not limited to, asthma, arthritis, allergic rhinitis, psoriasis, atopic dermatitis, inflammatory bowel diseases, Crohn's disease, and allergic rhinoconjunctitis. In still further embodiments, the cancer diseases to be treated include, but are not limited to, prostate cancer, breast cancer and colon cancer. In additional embodiments, the mammalian diseases to be treated include psychiatric disorders. Psychiatric disorders include, but are not limited to, depression, bipolar disorder, schizophrenia. In addition, the compositions of the invention can be used to maintain and/or enhance cognitive function.

Another embodiment of the present invention provides a method of treating a mammalian disease in a subject in need thereof by administration to the subject a therapeutically effective amount of a polyunsaturated fatty acid composition comprising at least 8% polyunsaturated fatty acids extracted from a microalgae, wherein the fatty acids can be (a) GLA in an amount of 1% to 10% of total fatty acids; (b) SDA in an amount of 5% to 50% of total fatty acids; (c) EPA in an amount of 2% to 30% of total fatty acids, and (d) DHA in an amount of 2% to 30% of total fatty acids. Further details on the amounts and ranges of polyunsaturated fatty acids, GLA, SDA, EPA and DHA in the compositions are as described above in the descriptions of the compositions.

An additional aspect of the invention provides a method of treating a mammalian disease in a subject in need thereof by administration to the subject a therapeutically effective amount of a composition comprising at least 5% SDA, the composition comprising either (a) a microalgal oil extracted from a cultivated microalgae biomass or (b) a microalgal biomass from a cultivated microalgae, wherein water is removed from microalgal biomass of (b) to achieve a solids content from about 5 to 100% weight percent. In some other embodiments, the microalgal oil and the microalgal biomass can be combined in the composition comprising 5% SDA. Further details on the amounts and ranges of SDA in the compositions are as described above in the descriptions of the compositions.

Subjects suitable to be treated according to the present invention include, but are not limited to, avian and mammalian subjects. Illustrative avians according to the present invention include chickens, ducks, turkeys, geese, quail, pheasant, ratites (e.g., ostrich), domesticated birds (e.g., parrots and canaries), and birds in ovo. Mammals of the present invention include, but are not limited to, canines, felines, bovines, caprines, equines, ovines, porcines, rodents (e.g. rats and mice), lagomorphs, primates (including non-human primates), humans, and the like, and mammals in utero. Any mammalian subject in need of being treated according to the present invention is suitable. According to some embodiments of the present invention, the mammal is a non-human mammal. In some embodiments, the mammal is a human subject. Mammalian subjects of both genders and at any stage of development (i.e., neonate, infant, juvenile, adolescent, adult) can be treated according to the present invention. Micro algae.

Any microalgae capable of producing a microalgal oil or microalgal biomass containing at least one polyunsaturated fatty acid from GLA, SDA, EPA, and DHA can be used in the processes, compositions, dietary supplements, and feed additives of the present invention. Thus, in some embodiments, the microalgae of the present invention is selected from the group consisting of Dinophyceae, Cryptophyceae, Trebouxiophyceae, Pinguiophyceae, and combinations thereof. In other embodiments, the microalgae of the invention are selected from the group consisting of Parietochloris spp., Rhodomonas spp., Cryptomonas spp., Parietochloris spp., Hemisebnis spp.; Porphyridium spp., Glossomastix spp., and combinations thereof. In further embodiments, the microalgae of the invention are selected from the group consisting of Parietochloris incise, Rhodomonas salina, Hemiselmis brunescens, Porphyridium cruentum and Glossomastix chrysoplasta, and combinations thereof. In still further embodiments, the microalgae of the invention is Rhodomonas salina.

In some embodiments of the invention, the microalgae can be a mixture of different microalgal species. In other embodiments, the microalgae is a single microalgal species. In some embodiments of the present invention, the microalgal fatty acids are provided as a microalgal oil. In other embodiments, the microalgal fatty acids are provided as a microalgal biomass.

Further, the microalgae of the invention include, but are not limited to, wild-type, mutant (naturally or induced) or genetically engineered microalgae.

Additionally, the microalgae of the present invention includes microalgae having cells with cell walls of reduced thickness as compared to the cells of wild-type microalgae, whereby the cell wall of reduced thickness improves extractability and/or bioavailability of the microalgal lipid fraction (e.g., improving the ease of digestibility of the microalgae and the ease of extractability of the microalgal oils from the cells of the microalgal biomass). Microalgae having cells with cell walls of reduced thickness as compared to the cells of wild-type microalgae can be naturally occurring, mutated and/or genetically engineered to have cell walls of reduced thickness as compared to wild-type strains. Thus, in one embodiment of the invention the microalgae is a microalgae having a cell wall of reduced thickness as compared to the wild-type microalgae, whereby said cell wall of reduced thickness improves extractability and/or bioavailability of the microalgal lipid fraction.

Methods of producing microalgae with reduced cell walls include those found in WO 2006/107736 A1, herein incorporated by reference in its entirety. Thus, the microalgae can be mutagenized with mutagens known to those of skill in the art including, but not limited to, chemical agents or radiation. In particular embodiments the chemical mutagens include, but are not limited to, ethyl methanesulfonate (EMS), methylmethane sulfonate (MMS), N-ethyl-N-nitrosourea (ENU), triethylmelamine (TEM), N-methyl-N-nitrosourea (MNU), procarbazine, chlorambucil, cyclophosphamide, diethyl sulfate, acrylamide monomer, melphalan, nitrogen mustard, vincristine, dimethylnitosamine, N-methyl-N′-nitro-Nitrosoguanidine (MNNG), nitrosoguanidine, 2-aminopurine, 7,12 diinethyl-benz(a)anthracene (DMBA), ethylene oxide, hexamethylphosphoramide, bisulfan, diepoxyalkanes (diepoxyoctane (DEO), diepoxybutane (BEB), and the like), 2-methoxy-6-chloro-9 [3-(ethyl-2-chlor-o-ethypaminopropylamino] acridine dihydrochloride (ICR-170), formaldehyde, and the like. Methods of radiation mutagenesis include, but are not limited to, x-rays, gamma-radiation, ultra-violet light, and the like.

Cell wall mutants can be selected for on the basis of increased sensitivity to detergents or by microscopic observation of alterations in cell wall thickness (WO 2006/107736 A1) or any other method known in the art to detect reduced cell wall thickness or reduced cell wall integrity.

The microalgae of the present invention can be cultured according to techniques described below in Example 1. In addition, the microalgae of the present invention can be cultured according to techniques known in the art including those techniques described by U.S. Pat. No. 5,244,921; U.S. Pat. No. 5,324,658; U.S. Pat. No. 5,338,673; U.S. Pat. No. 5,407,957; Mansour et al., J. Appl. Phycol. 17: 287-300 (2005); and Bigogno et al., Phytochemistry, 60: 497-503 (2002), the disclosures of which are to be incorporated by reference herein in their entirety.

Accordingly, in some embodiments the microalgae are cultured at a temperature in a range from 10° C. to 25° C. Thus, the microalgae can be cultured at a temperature of 10° C., 11° C., 12° C., 13° C., 14° C., 15° C., 16° C., 17° C., 18° C., 19° C., 20° C., 21° C., 22° C., 23° C., 24° C., 25° C., and the like. In other embodiments, the microalgae can be grown in ranges from 10° C. to 15° C., 10° C. to 20° C., 10° C. to 25° C., 12° C. to 15° C., 12° C. to 17° C., 12° C. to 20° C., 12° C. to 22° C., 12° C. to 24° C., 14° C. to 17° C., 14° C. to 19° C., 14° C. to 22° C., 14° C. to 25° C., 15° C. to 18° C., 15° C. to 20° C., 15° C. to 23° C., 15° C. to 25° C., 16° C. to 18° C., 16° C. to 19° C., 16° C. to 21° C., 16° C. to 23° C., 16° C. to 25° C., 17° C. to 19° C., 17° C. to 20° C., 17° C. to 23° C., 17° C. to 25° C., 18° C. to 20° C., 18° C. to 22° C., 18° C. to 23° C., 18° C. to 25° C., 19° C. to 21° C., 19° C. to 23° C., 19° C. to 25° C., 20° C. to 23° C., 20° C. to 25° C., 23° C. to 25° C., and the like. In a particular embodiment, the microalgae are grown at 14° C. In another embodiment, the microalgae are grown at 22° C.

In some embodiment, the microalgae are cultured at a light intensity in a range from 75 μmol m⁻² s⁻¹ to 150 μmol m⁻² s⁻¹. Accordingly, the microalgae can be grown at a light intensity of 75, 80, 85, 90, 95, 100, 105, 110, 115, 120,-125, 130, 135, 140, 145, 150}μmol m⁻² s⁻¹ In other embodiments, the microalgae can be grown at a light intensity in a range from 75 to 85 μmol m⁻² s⁻¹, 75 to 95 μmol m⁻² s⁻¹, 75 to 105 μmol m⁻² s⁻¹, 75 to 115 μmol m⁻²s⁻¹, 75 to 125 μumol m⁻² s⁻¹, 75 to 135 μmol m⁻² s⁻¹, 75 to 150 μmol m⁻² s⁻¹, 85 to 100 μmol m⁻² s⁻¹, 85 to 115 μmol m⁻² s⁻¹, 85 to 130 μmol m⁻² s⁻¹, 85 to 150 μmol m⁻² s⁻¹, 95 to 115 μmol m⁻² s⁻¹, 95 to 125 μmol m⁻² s⁻¹, 95 to 135 μmol m⁻² s⁻¹, 95 to 150 μmol m⁻² s⁻¹, 100 to 125 μmol m⁻² s⁻¹, 100 to 140 μmol m⁻² s⁻¹, 100 to 150 μmol m⁻² s⁻¹, 110 to 125 μmol m−2 s⁻¹, 110 to 135 μmol m⁻² s⁻¹, 110 to 150 μmol m⁻² s⁻¹, 120 to 130 μmol m⁻² s⁻¹, 120 to 140 μmol m⁻² s⁻¹, 120 to 150 μmol m⁻² s⁻¹, 130 to 140 μmol m² s⁻¹, 130 to 150 μmol m⁻² s⁻¹, 140 to 150 μmol m⁻² s⁻¹, and the like. In a particular embodiment, the microalgae are cultivated at a light intensity of 100 μmol m⁻² s⁻¹.

Following cultivation of the microalgae to the desired density, the microalgae are harvested using conventional procedures known to those of skill in the art and may include centrifugation, flocculation or filtration. The harvested microalgal cells or microalgal biomass can then be used directly as a fatty acid source or extracted to obtain microalgal oil comprising the fatty acids. In some embodiments in which the microalgal biomass is to be used directly, water is removed from the microalgal biomass to achieve a solids content from about 5 to 100 weight percent. In additional embodiments, a microalgal biomass that is to be used directly is comprised of microalgal cells further comprising cell walls that are at least partially disrupted to increase the extractability and/or bioavailability of the microalgal oil within the cells. The disruption of the microalgal cells can be carried out according to conventional techniques including, but not limited to, treating the cells with boiling water or by mechanical breaking such as grinding, pulverizing, sonication or the French press, or any other method known to those of skill in the art.

As stated above, in some embodiments, when the microalgal biomass is to be used directly, water is removed from the microalgal biomass to achieve a solids content from about 5 to 100%. Accordingly, water is removed from the microalgal biomass to achieve a solids content of 5%, 10%, 15%, 20%, 25%, 30%, 35%, 40%, 45%, 50%, 55%, 60%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, and the like. In additional embodiments, water is removed from the microalgal biomass to achieve a solids content in the range from about 5% to 50%, 5% to 60%, 5% to 70%, 5% to 80%, 5% to 90%, 5% to 95%, 10% to 30%, 10% to 40%, 10% to 50%, 10% to 60% 10% to 65%, 10% to 70%, 10% to 75%, 10% to 80%, 10% to 85%, 10% to 90%, 10% to 95%, 10% to 100%, 15% to 40%, 15% to 50%, 15% to 60%, 15% to 65%, 15% to 70%, 15% to 75%, 15% to 80%, 15% to 85%, 15% to 90%, 15% to 95%, 15% to 100%, 20% to 50%, 20% to 60%, 20% to 65%, 20% to 70%, 20% to 75%, 20% to 80%, 20% to 85%, 20% to 90%, 20% to 95%, 20% to 100%, 25% to 50%, 25% to 60%, 25% to 70%, 25% to 75%, 25% to 80%, 25% to 85%, 25% to 90%, 25% to 95%, 25% to 100%, 30% to 50%, 30% to 60%, 30% to 70%, 30% to 75%, 30% to 80%, 30% to 85%, 30% to 90%, 30% to 95%, 45% to 100%, 50% to 70%, 50% to 75%, 50% to 80%, 50% to 85%, 50% to 90%, 50% to 95%, 50% to 100%, 55% to 75%, 55% to 80%, 55% to 85%, 55% to 90%, 55% to 95%, 55% to 100%, 60% to 75%, 60% to 80%, 60% to 85%, 60% to 90%, 60% to 95%, 60% to 100%, 70% to 80%, 70% to 85%, 70% to 90%, 70% to 95%, 70% to 100%, 75% to 85%, 75% to 90%, 75% to 95%, 75% to 100%, 80% to 85%, 80% to 90%, 80% to 95%, 80% to 100%, 85% to 90%, 85% to 95%, 85% to 100%, 90% to 95%, 95% to 100%, and the like.

In some embodiments, the microalgal cells of the biomass can be disrupted or lysed and the microalgal oils extracted. The microalgal cells can be extracted wet or dry according to conventional techniques known to those of skill in the art, to produce a complex containing fatty acids. The disruption or lysis of the microalgal cells can be carried out according to conventional techniques including, but not limited to, treating the cells with boiling water or by mechanical breaking such as grinding, pulverizing, sonication or the French press, or any other method known to those of skill in the art. Extraction of the fatty acids from the lysed cells follow standard procedures used with microalgae and other organisms that are known to those of skill in the art, including, but not limited to, separating the liquid phase from the solid phase following cell lysis, extracting the fatty acids in the liquid phase by the addition of a solvent, evaporating the solvent, and recovering the polyunsaturated fatty acids obtained from the liquid phase of the lysed cells. See also, Bligh and Dyer, Can. J. Biochem. Physiol. 37:911-917 (1959); U.S. Pat. No. 5,397,591; U.S. Pat. No. 5,338,673, and U.S. Pat. No. 5,567,732; the disclosures herein incorporated by reference in their entirety.

Solvents that can be used for extraction include, but are not limited to, hexane, chloroform, ethanol, methanol, isopropanol, diethyl ether, dioxan, isopropyl ether, dichloromethane, tetrahydrofuran, and combinations thereof . In a further embodiment the microalgal cells can be extracted using supercritical fluid extraction with solvents such as CO₂ or NO. Extraction techniques using supercritical extraction are known to those of skill in the art and are described in McHugh et al. Supercritical Fluid Extraction, Butterworth, 1986, herein incorporated by reference in its entirety.

In the processes, compositions, food products, dietary supplements, feed additives and the like, of the present invention, the polyunsaturated fatty acids may be provided in the foiiii of free fatty acids, cholesterol esters, salt esters, fatty acid esters, monoglycerides, diglycerides, triglycerides, diacylglycerols, monoglycerols, sphingophospholipids, sphingoglycolipids, or any combination thereof. In some embodiments of the present invention, the fatty acids are provided in the Rolm of triglycerides. In other embodiments, the fatty acids are not provided in the form of phospholipids (e.g., are provided in a non phospholipid form).

The GLA of the present invention can be supplemented with additional GLA obtained from other sources, including, but not limited to, plants. Thus, the GLA of the present invention can be supplemented with GLA obtained from plant sources that include, but are not limited to, borage, black currant, echium, and primrose. In particular embodiments, the supplemental GLA is from borage or borage oil. In some embodiments, the microalgal GLA is supplemented with additional GLA from microalgal sources. In other embodiments, the GLA of the invention is not supplemented.

### Polyunsaturated Fatty Acid Compositions, Food Products and Animal Feed Additives.

The present invention further provides compositions made by the processes of the invention as described above. Accordingly, in some embodiments of the invention a polyunsaturated fatty acid composition is provided, the polyunsaturated fatty acid composition comprising at least 8% polyunsaturated fatty acids; the composition comprising at least one fatty acid extracted from a microalgae, wherein (a) GLA is in an amount of 1% to 10% of total fatty acids; (b) SDA is in an amount of 5% to 50% of total fatty acids; (c) EPA is in an amount of 2% to 30% of total fatty acids, and (d) DHA is in an amount of 2% to 30% of total fatty acids.

In some embodiments, the polyunsaturated fatty acid composition comprises polyunsaturated fatty acids at a concentration in a range from 5% to 35%. Thus, the polyunsaturated fatty acid composition can comprise polyunsaturated fatty acids at a concentration of 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, and the like. In other embodiments, the polyunsaturated fatty acid composition can comprise polyunsaturated fatty acids at a concentration in a range from 5% to 7%, 5% to 8%, 5% to 10%, 5% to 12%, 5% to 15%, 5% to 20%, 5% to 25%, 5% to 30%, 6% to 8%, 6% to 10%, 6% to 12%, 6% to 15%, 6% to 20%, 6% to 25%, 6% to 35%, 7% to 9%, 7% to 11%, 7% to 13%, 7% to 14%, 7% to 15%, 7% to 20%, 7% to 25%, 7% to 30%, 7% to 35%, 8% to 10%, 8% to 12% 8% to 14%, 8% to 15%, 8% to 20%, 8% to 25%, 8% to 35%, 9% to 11%, 9% to 13%, 9% to 15%, 9% to 20%, 9% to 25%, 9% to 30%, 9% to 35%, 10% to 12%, 10% to 13%, 10% to 14%, 10% to 15%, 10% to 20%, 10% to 25%, 10% to 30%, 10% to 35%, 15% to 20%, 15% to 25%, 15% to 30%, 20% to 25%, 20% to 30%, 20% to 35%, 25% to 30%, 25% to 35%, 30% to 35%, and the like. In one embodiment, the polyunsaturated fatty acid composition comprises polyunsaturated fatty acids at a concentration of at least 8%.

According to the present invention, the amount of GLA that can be included in the composition is in a range from 1% to 10% of total fatty acids. Thus, the GLA can be included in the composition in an amount of total fatty acids of 1%, 2%, 3%, 4%, 5%, 6%, 7%, 8%, 9%, 10%, and the like. In other embodiments, the GLA can be included in the composition in an amount of total fatty acids in a range from 1% to 3%, 1% to 5%, 1% to 7%, 1% to 9%, 2% to 4%, 2% to 6%, 2% to 8%, 2% to 10%, 3% to 5%, 3% to 7%, 3% to 9%, 3% to 10%, 4% to 6%, 4% to 8%, 4% to 10%, 5% to 7%, 5% to 8%, 5% to 9%, 5% to 10%, 6% to 8%, 6% to 9%, 6% to 10%, 7% to 9%, 7% to 10%, 8% to 10%, 9% to 10%, and the like.

In some embodiments, the amount of SDA that is included in the composition of the present invention is in a range from 5% to 50% of total fatty acids. Thus, the SDA can be provided in the composition in an amount of total fatty acids of 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, and the like. In other embodiments, the SDA can be included in the composition in an amount of total fatty acids in a range from 5% to 10%, 5% to 15%, 5% to 20%, 5% to 25%, 5% to 30%, 5% to 35%, 5% to 40%, 5% to 45%, 5% to 50%, 10% to 15%, 10% to 20%, 10% to 25%, 10% to 30%, 10% to 35%, 10% to 40%, 10% to 45%, 10% to 50%, 20% to 25%, 20% to 30%, 20% to 35%, 20% to 40%, 20% to 45%, 20% to 50%, 25% to 30%, 25% to 35%, 25% to 40%, 25% to 45%, 25% to 50%, 30% to 35%, 30% to 40%, 30% to 45%, 30% to 50%, 35% to 40%, 35% to 45%, 35% to 50%, 40% to 45%, 40% to 50%, 45% to 50%, and the like.

In other embodiments, the EPA can be included in the composition in a range from 2% to 30% of total fatty acids. Thus, the EPA can be provided in the composition in an amount of total fatty acids of 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, and the like. In other embodiments, the EPA can be included in the composition in an amount of percent of total fatty acids in a range from 1% to 5%, 1% to 10%, 1% to 15%, 1% to 20%, 1% to 25%, 1% to 30%, 5% to 10%, 5% to 15%, 5% to 20%, 5% to 25%, 5% to 30%, 10% to 15%, 10% to 20%, 10% to 25%, 10% to 30%, 15% to 20%, 15% to 25%, 15% to 30%, 20% to 25%, 20% to 30%, 25% to 30%, and the like.

In some embodiments of the present invention, the DHA can be included in the composition in a range from 2% to 30% of total fatty acids. Thus, the DHA can be provided in the composition in an amount of total fatty acids of 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, and the like. In other embodiments, the DHA can be included in the composition in an amount of total fatty acids in a range from 1% to 5%, 1% to 10%, 1% to 15%, 1% to 20%, 1% to 25%, 1% to 30%, 5% to 10%, 5% to 15%, 5% to 20%, 5% to 25%, 5% to 30%, 10% to 15%, 10% to 20%, 10% to 25%, 10% to 30%, 15% to 20%, 15% to 25%, 15% to 30%, 20% to 25%, 20% to 30%, 25% to 30%, and the like.

The present invention further provides a composition comprising at least 5% SDA, the composition comprising either: (a) a microalgal oil extracted from a cultivated microalgae biomass or (b) a microalgal biomass from a cultivated microalgae, wherein water is removed from microalgal biomass to achieve a solids content from about 5 to 100% weight percent. In some embodiments, the SDA is not in a phospholipid form.

In some embodiments, the SDA is present in the composition in an amount in a range from 2% to 10%. Thus, the SDA is present in the composition in an amount of 2%, 3%, 4%, 5%, 6%, 7%, 8%, 9%, 10%, and the like. In other embodiments, the SDA can be included in the composition in a range from 2% to 4%, 2% to 6%, 2% to 8%, 2% to 10%, 3% to 5%, 3% 25 to 7%, 3% to 9%, 3% to 10%, 4% to 6%, 4% to 8%, 4% to 10%, 5% to 7%, 5% to 8%, 5% to 9%, 5% to 10%, 6% to 8%, 6% to 9%, 6% to 10%, 7% to 9%, 7% to 10%, 8% to 10%, 9% to 10%, and the like. In some embodiments, the SDA is not in a phospholipid form.

In an additional embodiment, the present invention provides a food product comprising: (a) from 0.01-99.99 percent by weight of a composition comprising at least 8% polyunsaturated fatty acids, wherein the fatty acids are extracted from a microalgae, further wherein (i) GLA is in an amount of 1% to 10% of total fatty acids; (ii) SDA is in an amount of 5% to 50% of total fatty acids; (iii) EPA is in an amount of 2% to 30% of total fatty acids, and (iv) DHA is in an amount of 2% to 30% of total fatty acids; in combination with (b) from 99.99 to 0.01 percent by weight of at least one additional ingredient selected from the group consisting of proteins, carbohydrates and fiber, and combinations thereof.

In some embodiments, the food product of the invention can comprise polyunsaturated fatty acids at a concentration in a range from 5% to 35%. Thus, the food product can comprise polyunsaturated fatty acids at a concentration of 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, and the like. In other embodiments, the food product can comprise polyunsaturated fatty acids at a concentration in a range from 5% to 7%, 5% to 8%, 5% to 10%, 5% to 12%, 5% to 15%, 5% to 20%, 5% to 25%, 5% to 30%, 6% to 8%, 6% to 10%, 6% to 12%, 6% to 15%, 6% to 20%, 6% to 25%, 6% to 35%, 7% to 9%, 7% to 11%, 7% to 13%, 7% to 14%, 7% to 15%, 7% to 20%, 7% to 25%, 7% to 30%, 7% to 35%, 8% to 10%, 8% to 12%, 8% to 14%, 8% to 15%, 8% to 20%, 8% to 25%, 8% to 35%, 9% to 11%, 9% to 13%, 9% to 15%, 9% to 20%, 9% to 25%, 9% to 30%, 9% to 35%, 10% to 12%, 10% to 13%, 10% to 14%, 10% to 15%, 10% to 20%, 10% to 25%, 10% to 30%, 10% to 35%, 15% to 20%, 15% to 25%, 15% to 30%, 20% to 25%, 20% to 30%, 20% to 35%, 25% to 30%, 25% to 35%, 30% to 35%, and the like. In one embodiment, the food product comprises polyunsaturated fatty acids at a concentration of at least 8%.

According to the present invention, the amount of GLA that can be included in the food product is in a range from 1% to 10% of total fatty acids. Thus, the GLA can be included in the food product in an amount of total fatty acids of 1%, 2%, 3%, 4%, 5%, 6%, 7%, 8%, 9%, 10%, and the like. In other embodiments, the GLA can be included in the food product in an amount of total fatty acids in a range from 1% to 3%, 1% to 5%, 1% to 7%, 1% to 9%, 2% to 4%, 2% to 6%, 2% to 8%, 2% to 10%, 3% to 5%, 3% to 7%, 3% to 9%, 3% to 10%, 4% to 6%, 4% to 8%, 4% to 10%, 5% to 7%, 5% to 8%, 5% to 9%, 5% to 10%, 6% to 8%, 6% to 9%, 6% to 10%, 7% to 9%, 7% to 10%, 8% to 10%, 9% to 10%, and the like.

In some embodiments, the amount of SDA that is included in the food product of the present invention is in a range from 5% to 50% of total fatty acids. Thus, the SDA can be provided in the food product in an amount of total fatty acids of 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, and the like. In other embodiments, the SDA can be included in the food product in an amount of total fatty acids in a range from 5% to 10%, 5% to 15%, 5% to 20%, 5% to 25%, 5% to 30%, 5% to 35%, 5% to 40%, 5% to 45%, 5% to 50%, 10% to 15%, 10% to 20%, 10% to 25%, 10% to 30%, 10% to 35%, 10% to 40%, 10% to 45%, 10% to 50%, 20% to 25%, 20% to 30%, 20% to 35%, 20% to 40%, 20% to 45%, 20% to 50%, 25% to 30%, 25% to 35%, 25% to 40%, 25% to 45%, 25% to 50%, 30% to 35%, 30% to 40%, 30% to 45%, 30% to 50%, 35% to 40%, 35% to 45%, 35% to 50%, 40% to 45%, 40% to 50%, 45% to 50%, and the like.

In other embodiments, the EPA can be included in the food product in a range from 2% to 30% of total fatty acids. Thus, the EPA can be provided in the food product in an amount of total fatty acids of 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, and the like. In other embodiments, the EPA can be included in the food product in an amount of percent of total fatty acids in a range from 1% to 5%, 1% to 10%, 1% to 15%, 1% to 20%, 1% to 25%, 1% to 30%, 5% to 10%, 5% to 15%, 5% to 20%, 5% to 25%, 5% to 30%, 10% to 15%, 10% to 20%, 10% to 25%, 10% to 30%, 15% to 20%, 15% to 25%, 15% to 30%, 20% to 25%, 20% to 30%, 25% to 30%, and the like.

In some embodiments of the present invention, the DHA can be included in the food product in a range from 2% to 30% of total fatty acids. Thus, the DHA can be provided in the food product in an amount of total fatty acids of 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, and the like. In other embodiments, the DHA can be included in the food product in an amount of total fatty acids in a range from 1% to 5%, 1% to 10%, 1% to 15%, 1% to 20%, 1% to 25%, 1% to 30%, 5% to 10%, 5% to 15%, 5% to 20%, 5% to 25%, 5% to 30%, 10% to 15%, 10% to 20%, 10% to 25%, 10% to 30%, 15% to 20%, 15% to 25%, 15% to 30%, 20% to 25%, 20% to 30%, 25% to 30%, and the like.

Further embodiments of the invention provide a food product comprising: (a) from 0.01-99.99 percent by weight of a composition comprising at least 5% stearidonic acid (weight percent; w/w), the composition comprising either: (i) a microalgal oil extracted from a cultivated microalgae biomass or (ii) a microalgal biomass from a cultivated microalgae, wherein water is removed from microalgal biomass to achieve a solids content from about 5 to 100% weight percent; in combination with (b) from-99.99 to 0.01 percent by weight of at least one additional ingredient selected from the group consisting of proteins, carbohydrates and fiber, and combinations thereof. In some embodiments of the invention, the SDA is not in a phospholipid form.

In some embodiments, the SDA is present in the composition in an amount in a range from 2% to 10%. Thus, the SDA is present in the composition in an amount of 2%, 3%, 4%, 5%, 6%, 7%, 8%, 9%, 10%, and the like. In other embodiments, the SDA can be included in the composition in a range from 2% to 4%, 2% to 6%, 2% to 8%, 2% to 10%, 3% to 5to 7%, 3% to 9%, 3% to 10%, 4% to 6%, 4% to 8%, 4% to 10%, 5% to 7%, 5% to 8%, 5% to 9%, 5% to 10%, 6% to 8%, 6% to 9%, 6% to 10%, 7% to 9%, 7% to 10%, 8% to 10%, 9% to 10%, and the like.

In the present invention, the amount of the fatty acid composition in any of the food products described herein can be between 0.01% and 99.99% by weight of the food product. Thus, the amount of fatty acid composition in the food product can be 0.1%, 0.2%, 0.3%, 0.4%, 0.5%, 1%, 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, 99.5%, 99.7%, 99.8%, 99.9% and the like. In other embodiments, the amount of the fatty acid composition in the food product is in a range from 0.1% to 5%, 0.1% to 10%, 0.1% to 15%, 0.1% to 20%, 0.1% to 25%, 0.1% to 30%, 0.1% to 35%, 0.1% to 40%, 0.1% to 45%, 0.1% to 50%, 0.1% to 60%, 0.1% to 70%, 0.1% to 80%, 0.1% to 90%, 0.1% to 99%, 0.1% to 99.5%, 0.5% to 5%, 0.5% to 10%, 0.5% to 15%, 0.5% to 20%, 0.5% to 25%, 0.5 to 35%, 0.5% to 45%, 0.5% to 55%, 0.5% to 65%, 5% to 25%, 5% to 35%, 5% to 45%, 5% to 55%, 5% to 65%, 5% to 75%, 5% to 80%, 5% to 85%, 5% to 95%, 5% to 99%, 10% to 30%, 10% to 40% 10% to 50%, 10% to 60%, 10% to 70%, 10% to 75%, 10% to 80%, 10% to 85%, 10% to 95%, 10% to 99%, 10% to 99.9%, 15% to 35%, 15% to 45%, 15% to 55%, 15% to 65%, 15% to 75%, 15% to 85%, 15% to 95%, 15% to 99%, 15% to 99.9%, 20% to 40%, 20% to 50%, 20% to 60%, 20% to 70%, 20% to 75%, 20% to 80%, 20% to 85%, 20% to 95%, 20% to 99%, 25% to 40%, 25% to 50%, 25% to 60%, 25% to 70%, 25% to 75%, 25% to 80%, 25% to 85%, 25% to 95%, 25% to 99%, 30% to 50%, 30% to 55%, 30% to 60%, 30% to 65%, 30% to 70%, 30% to 75%, 30% to 80%, 30% to 85%, 30% to 90%, 30% to 95%, 30% to 99%, 35% to 50%, 35% to 55%, 35% to 60%, 35% to 65%, 35% to 70%, 35% to 75%, 35% to 80%, 35% to 85%, 35% to 90%, 35% to 95%, 35% to 99%, 40% to 50%, 40% to 55%, 40% to 60%, 40% to 65%, 40% to 70%, 40% to 75%, 40% to 80%, 40% to 85%, 40% to 90%, 40% to 95%, 40% to 99%, 45% to 60%, 45% to 65%, 45% to 70%, 45% to 75%, 45% to 80%, 45% to 85%, 45% to 90%, 45% to 95%, 45% to 99%, 50% to 60%, 50% to 65%, 50% to 70%, 50% to 75%, 50% to 80%, 50% to 85%, 50% to 90%, 50% to 95%, 50% to 99%, 55% to 65%, 55% to 70%, 55% to 75%, 55% to 80%, 55% to 85%, 55% to 90%, 55% to 95%, 55% to 99%, 60% to 70%, 60% to 75%, 60% to 80%, 60% to 85%, 60% to 90%, 60% to 95%, 60% to 99%, 65% to 80%, 65% to 85%, 65% to 90%, 65% to 95%, 65% to 99%, 70% to 80%, 70% to 85%, 70% to 90%, 70% to 95%, 70% to 99%, 75% to 85%, 75% to 90%, 75% to 95%, 75% to 99%, 80% to 90%, 80% to 95%, 80% to 99%, 85% to 90%, 85% to 95%, 85% to 99%, 90% to 95%, 90% to 99%, 95% to 99%, and the like.

The present invention further provides a liquid dietary supplement for diminishing symptoms of inflammatory disorders, said supplement consisting essentially of: 19 weight percent water; 25 weight percent sucrose; 35 weight percent oils, wherein the oils are GLA and SDA from a microalgae; 15 weight percent flavoring; and 5 weight percent glycerin.

In further embodiments, the water can be in a range from 10-30% weight percent. Thus, the water can be present in an amount of 10%, 11%, 12%, 13%, 14%, 15%, 16%, 17%, 18%, 19%, 20%, 21%, 22%, 23%, 24%, 25%,26%, 27%, 28%, 29%, 30%, and additional embodiments, the sucrose is present in an amount in a range from 10% to 40%. Thus, the sucrose can be present in an amount of 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40% and the like.

In still further embodiments, the oils can be present in an amount in a range from 20% to 50% (weight percent; w/w). Thus, the oils can be present in an amount of 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, and the like. In some embodiments, the flavoring can be present in an amount in a range from 5%-25%. Thus, the flavoring can be present in an amount of 5%, 6%, 7%, 8%, 9%, 10%, 11%, 12%, 13%, 14%, 15%, 16%, 17%, 18%, 19%, 20%, 21%, 22%, 23%, 24%, 25%, and the like. In other embodiments, the glycerin can be present in a range from 1%-20%. Thus, the glycerin can be present in an amount of 1%, 2%, 3%, 4%, 5%, 6%, 7%, 8%, 9%, 10%, 11%, 12%, 13%, 14%, 15%, 16%, 17%, 18%, 19%, 20%, and the like.

The liquid dietary supplement can further comprise less than 1 weight percent minor ingredients selected from antioxidants, preservatives, colorants, stabilizers, emulsifiers or a combination thereof.

In some embodiments, the weight ratio of GLA to SDA in the liquid dietary supplement can be in a range from 6:1 to 1:6. Thus, the weight ratio of GLA to SDA can be 6.0:1.0, 5.0:1.0, 4.0:1.0, 3.0:1.0, 3.0:0.5, 2.5:1.5, 2.5:0.5, 2.0:1.0, 2.0:0.5, 1.0:1.0, 1.0:2.0, 1.0:3.0, 1.0:4.0, 1.0:5.0, 1.0:6.0, and the like.

The present invention further provides animal feed additives made by the processes of the invention described herein. Thus, in some embodiments of the invention an animal feed additive is provided wherein the animal feed additive comprises polyunsaturated fatty acids collected from microalgae either in the form of: a) a microalgal oil extracted from a cultivated microalgae biomass or (b) a microalgal biomass from a cultivated microalgae, wherein water is removed from microalgal biomass to achieve a solids content from about 5 to 100% weight percent.

In further embodiments, the fatty acids collected from the microalgae for the animal feed additive are short chain omega-3 fatty acids.

Additionally provided herein is an animal feed additive comprising at least 8% polyunsaturated fatty acids; the additive comprising fatty acids extracted from a microalgae, wherein: (a) GLA is in an amount of 1% to 10% of total fatty acids; (b) SDA is in an amount of 5% to 50% of total fatty acids; (c) EPA is in an amount of 2% to 30% of total fatty acids, and (d) DHA is in an amount of 2% to 30% of total fatty acids.

In some embodiments, the animal feed additive comprises polyunsaturated fatty acids at a concentration in a range of 5% to 15%. Thus, the animal feed additive can comprise polyunsaturated fatty acids at a concentration of 5%, 6%, 7%, 8%, 9%, 10%, 11%, 12%, 13%, 14%, 15%, and the like. In other embodiments, the animal feed additive can comprise polyunsaturated fatty acids at a concentration in a range from 5% to 7%, 5% to 8%, 5% to 10%, 5% to 12%, 5% to 15%, 6% to 8%, 6% to 10%, 6% to 12%, 6% to 15%, 7% to 9%, 7% to 11%, 7% to 13%, 7% to 14%, 7% to 15%, 8% to 10%, 8% to 12%, 8% to 14%, 8% to 15%, 9% to 11%, 9% to 13%, 9% to 15%, 10% to 12%, 10% to 13%, 10% to 14%, 10% to 15%, and the like. In one embodiment, the animal feed additive comprises polyunsaturated fatty acids at a concentration of at least 8%.

According to the present invention, the amount of GLA in the animal feed additive is in a range from 1% to 10% of total fatty acids. Thus, the GLA in the animal feed additive can be in an amount of total fatty acids of 1%, 2%, 3%, 4%, 5%, 6%, 7%, 8%, 9%, 10%, and the like. In other embodiments, the GLA in the animal feed additive can be in an amount of total fatty acids in a range from 1% to 3%, 1% to 5%, 1% to 7%, 1% to 9%, 2% to 4%, 2% to 6%, 2% to 8%, 2% to 10%, 3% to 5%, 3% to 7%, 3% to 9%, 3% to 10%, 4% to 6%, 4% to 8%, 4% to 10%, 5% to 7%, 5% to 8%, 5% to 9%, 5% to 10%, 6% to 8%, 6% to 9%, 6% to 10%, 7% to 9%, 7% to 10%, 8% to 10%, 9% to 10%, and the like.

In some embodiments, the amount of SDA in the animal feed additive of the present invention is in a range from 5% to 50% of total fatty acids. Thus, the animal feed additive can comprise SDA in an amount of total fatty acids of 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%,29%,30%,31%,32%, 33%, 34%, 35%, 36%, 37%, 38%, 39%, 40%, 41%, 42%, 43%, 44%, 45%, 46%, 47%, 48%, 49%, 50%, and the like. In other embodiments, the SDA in the animal feed additive is in an amount of total fatty acids in a range from 5% to 10%, 5% to 15%, 5% to 20%, 5% to 25%, 5% to 30%, 5% to 35%, 5% to 40%, 5% to 45%, 5% to 50%, 10% to 15%, 10% to 20%, 10% to 25%, 10% to 30%, 10% to 35%, 10% to 40%, 10% to 45%, 10% to 50%, 20% to 25%, 20% to 30%, 20% to 35%, 20% to 40%, 20% to 45%, 20% to 50%, 25% to 30%, 25% to 35%, 25% to 40%, 25% to 45%, 25% to 50%, 30% to 35%, 30% to 40%, 30% to 45%, 30% to 50%, 35% to 40%, 35% to 45%, 35% to 50%, 40% to 45%, 40% to 50%, 45% to 50%, and the like.

In other embodiments, the EPA in the animal feed additive can be in a range from 2% to 30% of total fatty acids. Thus, the EPA in the animal feed additive is in an amount of total fatty acids of 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%,30%, and the like.

In other embodiments, the EPA in the animal feed additive is in an amount of percent of total fatty acids in a range from 1% to 5%, 1% to 10%, 1% to 15%, 1% to 20%, 1% to 25%, 1% to 30%, 5% to 10%, 5% to 15%, 5% to 20%, 5% to 25%, 5% to 30%, 10% to 15%, 10% to 20%, 10% to 25%, 10% to 30%, 15% to 20%, 15% to 25%, 15% to 30%, 20% to 25%, 20% to 30%, 25% to 30%, and the like.

In some embodiments of the present invention, the DHA in the animal feed additive is in a range from 2% to 30% of total fatty acids. Thus, the DHA in the animal feed additive is in an amount of total fatty acids of 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, and the like. In other embodiments, the DHA is in the animal feed additive in an amount of total fatty acids in a range from 1% to 5%, 1% to 10%, 1% to 15%, 1% to 20%, 1% to 25%, 1% to 30%, 5% to 10%, 5% to 15%, 5% to 20%, 5% to 25%, 5% to 30%, 10% to 15%, 10% to 20%, 10% to 25%, 10% to 30%, 15% to 20%, 15% to 25%, 15% to 30%, 20% to 25%, 20% to 30%, 25% to 30%, and the like.

In other embodiments of the present invention further comprise animal products produced by feeding to an animal the animal feed additives described herein. Therefore, one aspect of the invention includes an animal product produced by feeding to an animal an animal feed additive comprising polyunsaturated fatty acids collected from microalgae either in the faun of: (a) a microalgal oil extracted from a cultivated microalgae biomass or (b) a microalgal biomass from a cultivated microalgae, wherein water is removed from microalgal biomass to achieve a solids content from about 5 to 100% weight percent.

Still other aspects of the invention provide an animal product produced by feeding to an animal an animal feed additive comprising at least 8% polyunsaturated fatty; the additive comprising fatty acids extracted from a microalgae, wherein the microalgal fatty acid extract comprises (a) GLA in an amount of 1% to 10% of total fatty acids; (b) SDA in an amount of 5% to 50% of total fatty acids; (c) EPA in an amount of 2% to 30% of total fatty acids; and (d) DHA in an amount of 2% to 30% of total fatty acids.

An animal product of the present invention includes, but is not limited to, eggs, milk, or meat.

The compositions of the present invention as described herein may be used as a complete food product, as a component of a food product, as a dietary supplement or as part of a dietary supplement, as a feed additive and may be either in liquid, semisolid or solid form. The compositions of the present invention as described herein additionally may be in the form of a pharmaceutical composition. The compositions, dietary supplements, food products, baby food products, feed additives, and/or pharmaceutical compositions of the present invention may advantageously be utilized in methods for promoting the health of an individual.

As indicated above, the compositions may be in liquid, semisolid or solid form. For example, the compositions may be administered as tablets, gel packs, capsules, gelatin capsules, flavored drinks, as a powder that can be reconstituted into such a drink, cooking oil, salad oil or dressing, sauce, syrup, mayonnaise, margarine or the like. Furthermore, the food product, dietary supplements, and the like, of the present invention can include, but are not limited to, dairy products, baby food, baby formula, beverages, bars, a powder, a food topping, a drink, a cereal, an ice cream, a candy, a snack mix, a baked food product and a fried food product. Beverages of the invention include but are not limited to energy drinks, nutraceutical drinks, smoothies, sports drinks, orange juice and other fruit drinks. A bar of the present invention includes, but is not limited to, a meal replacement, a nutritional bar, a snack bar and an energy bar, an extruded bar, and the like. Dairy products of the invention include, but are not limited to, including but not limited to yogurt, yogurt drinks, cheese and milk.

The food products or dietary supplements of the present invention may further comprise herbals, herbal extracts, fungal extracts, enzymes, fiber sources, minerals, and vitamins. The microalgal oils and microalgal biomass of the present invention may be used in the compositions of the invention for both therapeutic and non-therapeutic uses. Thus, the compositions, food products and animal feed additives of the present invention may be used for therapeutic or non-therapeutic purposes.

Compositions intended for oral administration may be prepared according to any known method for the manufacture of dietary supplements or pharmaceutical preparations, and such compositions may include at least one additive selected from the group consisting of taste improving substances, such as sweetening agents or flavoring agents, stabilizers, emulsifiers, coloring agents and preserving agents in order to provide a dietetically or phanuaceutically palatable preparation. Vitamins, minerals and trace element from any physiologically acceptable source may also be included in the composition of the invention.

A pharmaceutical composition of the present invention comprises the said compositions of the present invention in a therapeutically effective amount. The compositions may additionally comprise prescription medicines or non-prescription medicines. The combinations may advantageously produce one or more of the following effects: (1) additive and/or synergistic benefits; (2) reduction of the side effects and/or adverse effects associated with use of the prescription medicine in the absence of the said formulations; and/or (3) the ability to lower the dosage of the prescription medicine in comparison to the amount of prescription medicine needed in the absence of the said formulations.

The active agents of the present invention can be prepared in the form of their pharmaceutically acceptable salts. As understood by one of skill in the art, pharmaceutically acceptable salts are salts that retain the desired biological activity of the parent compound and do not impart undesired toxicological effects. Examples of such salts are (a) acid addition salts formed with inorganic acids, for example, hydrochloric acid, hydrobromic acid, sulfuric acid, phosphoric acid, nitric acid and the like; and salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; (b) salts fowled from elemental anions such as chlorine, bromine, and iodine; and (c) salts derived from bases, such as ammonium salts, alkali metal salts such as those of sodium and potassium, alkaline earth metal salts such as those of calcium and magnesium, and salts with organic bases such asisopropylamine, trimethylamine, histidine, dicyclohexylamine and N-methyl-D-glucamine.

The active agents can be formulated for administration in accordance with known pharmacy techniques. See, e.g., Remington, The Science And Practice of Pharmacy (9th Ed. 1995). In the manufacture of a pharmaceutical composition according to the present invention, the active agents (including the physiologically acceptable salts thereof) is typically admixed with, inter alia, an acceptable carrier. The carrier must, of course, be acceptable in the sense of being compatible with any other ingredients in the formulation and must not be deleterious to the subject. The carrier can be a solid or a liquid, or both, and can be formulated with the active agent as a unit-dose formulation, for example, a tablet, which can contain from 0.01% or 0.5% to 95% or 99%, or any value between 0.01% and 99%, by weight of the active agent. One or more active agents can be incorporated in the compositions of the invention, which can be prepared by any of the well-known techniques of pharmacy, comprising admixing the components, optionally including one or more accessory ingredients. Moreover, the carrier can be preservative free, as described herein above.

In some embodiments, the active agents comprise a lower limit ranging from about 0.01, 0.02, 0.03, 0.04, 0.05, 0.06, 0.07, 0.08, 0.09, 1, 2, 3, 4, 5, 6, 7, 8, 9, and 10% to an upper limit ranging from about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 and 100% by weight of the composition. In some embodiments, the active agent includes from about 0.05% to about greater than 99% by weight of the composition.

The pharmaceutical compositions according to embodiments of the present invention are generally formulated for oral and topical (i.e., skin, ocular and mucosal surfaces) administration, with the most suitable route in any given case depending on the nature and severity of the condition being treated and on the nature of the particular active agent which is being used.

Formulations suitable for oral administration can be presented in discrete units, such as capsules, cachets, lozenges, or tablets, each containing a predetemined amount of the active compound; as a powder or granules; as a solution or a suspension in an aqueous or non-aqueous liquid; or as an oil-in-water or water-in-oil emulsion. Such formulations can be prepared by any suitable method of pharmacy, which includes bringing into association the active compound and a suitable carrier (which can contain one or more accessory ingredients as noted above). In general, the formulations of the invention are prepared by uniformly and intimately admixing the active compound with a liquid or finely divided solid carrier, or both, and then, if necessary, shaping the resulting mixture. For example, a tablet can be prepared by compressing or molding a powder or granules containing the active compound, optionally with one or more accessory ingredients. Compressed tablets can be prepared by compressing, in a suitable machine, the compound in a free-flowing form, such as a powder or granules optionally mixed with a binder, lubricant, inert diluent, and/or surface active/dispersing agent(s). Molded tablets can be made by molding, in a suitable machine, the powdered compound moistened with an inert liquid binder.

Further, formulations suitable for topical administration can be in the form of cremes and liquids including, for example, syrups, suspensions or emulsions, inhalants, sprays, mousses, oils, lotions, ointments, gels, solids and the like.

Suitable pharmaceutically acceptable topical carriers include, but are not limited to, water, glycerol, alcohol, propylene glycol, fatty alcohols, triglycerides, fatty acid esters, and mineral oils. Suitable topical cosmetically acceptable carriers include, but are not limited to, water, petroleum jelly, petrolatum, mineral oil, vegetable oil, animal oil, organic and inorganic waxes, such as microcrystalline, paraffin and ozocerite wax, natural polymers, such as xanthanes, gelatin, cellulose, collagen, starch or gum arabic, synthetic polymers, alcohols, polyols, and the like. Preferably, because of its non-toxic topical properties, the pharmaceutically and/or cosmetically-acceptable carrier is substantially miscible in water. Such water miscible carrier compositions can also include sustained or delayed release carriers, such as liposomes, microsponges, microspheres or microcapsules, aqueous based ointments, water-in-oil or oil-in-water emulsions, gels and the like.

The pharmaceutically acceptable compounds of the invention will normally be administered to a subject in a daily dosage regimen. For an adult subject this may be, for example, an oral dose of GLA between 0.1 gram and 15 grams. In further embodiments, an oral dose of GLA can be between 0.5 gram and 10 grams. In still further embodiments, an oral dose of GLA can be between 0.5 grams and 3 grams. In other embodiments, an oral dose of SDA can be between 0.1 g and 10 grams. In additional embodiments, an oral dose of SDA can be between 0.25 grams and 5 grams. In yet additional embodiments, an oral dose of SDA can be between 0.25 grams and 3 grams. In addition, some embodiments of the invention can optionally include an oral dose of EPA or DHA between about 0.1 g and about 15 g.

The pharmaceutical compositions may be administered 1 to 4 times per day. Thus in particular embodiments, compositions are contemplated comprising a 1:1 (w/w) ratio of GLA:EPA, wherein there may be 1, 2, 3, 4, 5, 6, 7, 8, 9, or 10 grams of GLA. In other embodiments there may be a 2:1 ratio of (w/w) ratio of GLA:EPA, wherein there may be 1, 2, 3, 4, 5, 6, 7, 8, 9, 10, 11, 12, 13 14 or 15 grams of GLA. Of course, the ratio of GLA:EPA administered may be varied from that disclosed herein above. For example, any amount of EPA including 0.1, 0.25, 0.5, 1, 2, 3, 4, 5, 6, 7, 8, 9 or 10 grams of EPA may be administered with any amount of GLA including 1, 2, 3, 4, 5, 6, 7, 8, 9, 10, 11, 12, 13, 14, or 15 grams of GLA. Such amounts of either supplement may be admixed in one composition or may be in distinct compositions.

The present invention will now be described with reference to the following example. It should be appreciated that this example is for the purpose of illustrating aspects of the present invention, and does not limit the scope of the invention as defined by the claims.

### EXAMPLES

### Example 1

### Culture Conditions

Rhodomonas salina cells were maintained in 125-ml flasks containing 50 ml of growth media (see below) at room temperature with continuous irradiance of 50 μmol m⁻² s⁻¹. Culture flasks were under constant shaking at 100 rpm, using a shaking table.

For all experiments, illumination was provided with white fluorescent bulbs (40 watt), various light intensities were achieved by changing the numbers of light bulbs or by adjusting the distance between the culture flasks and the light bulbs. For temperature experiments, culture flasks were incubated in a water bath at temperatures between 14° C. to 34° C. The temperature in the water bath was controlled by an electrical heating rod (Aquatic Ecosystem, Apopka, Fla.) at 22° C., 28° C., or 34° C., respectively. Compressed air enriched with 1-2% CO₂ was used to mix the cultures, as well as to facilitate gas (0₂ and CO₂) exchange and liquid mass transfer.

The growth medium used was the following f/2 Medium composition:

| | | Molar Concentration in |
|---|---|---|
| | Component | Final Medium |
| Macro-nutrients | NaNO₃ | 8.83 × 10⁻¹M |
| | NaH₂PO₄ H₂O | 3.63 × 10⁻⁵M |
| | Na₂SiO₃ 9H₂O* | 1.07 × 10⁻⁴M* |
| Micro-nutrients | FeCl₃ 6H₂O | 1 × 10⁻⁵M |
| | Na₂EDTA 2H₂O | 1 × 10⁻⁵M |
| | CuSO₄ SJ₂O | 4 × 10⁻⁵M |
| | Na₂MoO₄ 2H₂O | 3 × 10⁻⁸M |
| | ZnSO₄ 7H₂O | 8 × 10⁻⁸M |
| | CuCl₂ 6H₂O | 5 × 10⁻⁸M |
| | MnCl₂ 4H₂O | 9 × 10⁻⁷M |
| Vitamin Mix | Vitamin B₁₂ | 1 × 10⁻¹⁰M |
| | (cyanocobalamin) | |
| | Biotin | 2 × 10⁻⁹M |
| | Thiamine HCl | 3 × 10⁻⁷M |

All nutrient components were finally dissolved either in 1 liter filtered natural seawater or artificial seawater made up of 3.4% sea salt. The seawater was collected from Institute of Marine Sciences at UNC—Chapel Hill, Morehead City, N.C. The sea salt was purchased from Aquatic Ecosystem Inc. (Apopka, Fla.). The stock solutions for macro-nutrients, micro-nutrients, or vitamin mix were prepared separately and mix together before use. For axenic media preparation, the mixed media were autoclaved.

### Example 2

### Growth Measurement

The specific growth rate was measured by cell count, optical density of 550 mn (O.D. 550), chlorophyll concentration, or dry weight.

Cell counts: A one ml of culture suspension was withdrawn daily. Microalgal cells were fixed with Lugol's solution and counted with a haemocytometer. Cell concentration is expressed as total number of cells per milliliter of culture volume.

Dry weight analysis: A one to ten ml culture sample was filtered through a pre-dried, weighed Whatman GF/C filter paper. Cells on the filter paper were washed three times with 3.4% ammonia bicarbonate to remove the salt. The filter paper containing algal cells was dried overnight in an oven at 100° C. The ammonia bicarbonate evaporated during this process. The difference between the final weight and the weight before filtration was the dry weight of the sample (Lu et al., J. Phycol. 30: 829-833 (1994)).

O.D. 550: A one ml culture suspension was withdrawn daily to monitor the optical density at 550 nm using a Genesys 10V is spectrophotometer (Thermo Electron Corp.).

Chlorophyll & carotenoids: One-half ml to five ml culture sample was harvested by filtration on Whatman GF/C filter paper. One ml of 100% methanol was used to extract pigments overnight at 4° C. The supernatant was collected after centrifugation and pigments determined by absorption spectroscopy. The following equations were used to calculate chlorophyll and carotenoid content: Chl-α (μgmL⁻¹)=13.9 A₆₆₅; Total carotenoids (μgmL⁻¹)=4A₄₈₀ (Montero et al., Botanica Marina 45: 305-315 (2002)).

The specific growth rate was calculated using the following formula:

μ(d⁻¹)=(LnN₂−LnN₁)/(t₂−t₁)

Where t₁ and t₂ represent different time points, and N1 and N2 represent chlorophyll concentration, O.D. 550, dry weight or cell concentration at time t₁ and time t₂, respectively.

### Example 3

### Fatty Acids Extraction and Measurement

Cells were harvested by filtration on Whatman GF/C filter paper. Total lipids were extracted according to the method of Bligh and Dyer (Bligh, E. and W. Dyer, Can. J. Biochem. Physiol. 37: 911-917 (1959).

Fatty acids methyl ester analysis was performed using an Agilent 6890 GC equipped with a split/splittless injector at 230° C., a flame ionization detector at 260° C., an autosampler (Agilent Technologies, Waldbronn, Germany) and a CP SIL 88 column (100 m, 0.25 mm, 0.2.25 m film thickness, Varian, Datuistadt, Germany). Hydrogen was used as carrier gas at constant flow rate of 1 ml/min. The temperature of the GC oven was set to 70 ° C. for 3 min, increased at 8° C./min to 180° C., held for 2 min, increased at 4° C./min to 210° C., held for 4 min, increased at 2° C./min to a final temperature of 240° C. and held for 25 min. HP Chemstation software (Rev. A.08.03) was used for data analysis. The sample was injected using a split ratio of 1:10.

### Example 4

### Cytotoxicity Assay

The method for determining cytotoxicity was modified according to Meyer et al. (Planta Med. 45, 31-34 (1982)). Briefly, algal cells were tested at a concentration of 5×106 cells/ml in triplicates using a 96-well microplate. Brine shrimp eggs (Artemia salina Leach) were purchased in a local pet store and hatched in artificial seawater (solution of 3.4% sea salt) at room temperature. After 24 hours, the larvae (nauplii) were collected. A suspension of 8-12 nauplii (100 μl) was added to each well containing algal cells and the microplate was covered and incubated for 24-72 hours at room temperature. During this period, the number of dead nauplii in each well was counted using a binocular microscope (10×). The survival rate of the nauplii was used as the indicator for the toxicity of the algal species tested.

### Example 5

### Fatty Acid Profiles of Rhodomonas salina and Amphidinium carterae

The microalgae were cultivated in 125 ml flasks with f/2 medium under a light intensity of 50 μL mol m⁻² s⁻¹ at room temperature. After one week, cells were harvested by filtration and fatty acid compositions were analyzed by gas chromatography.

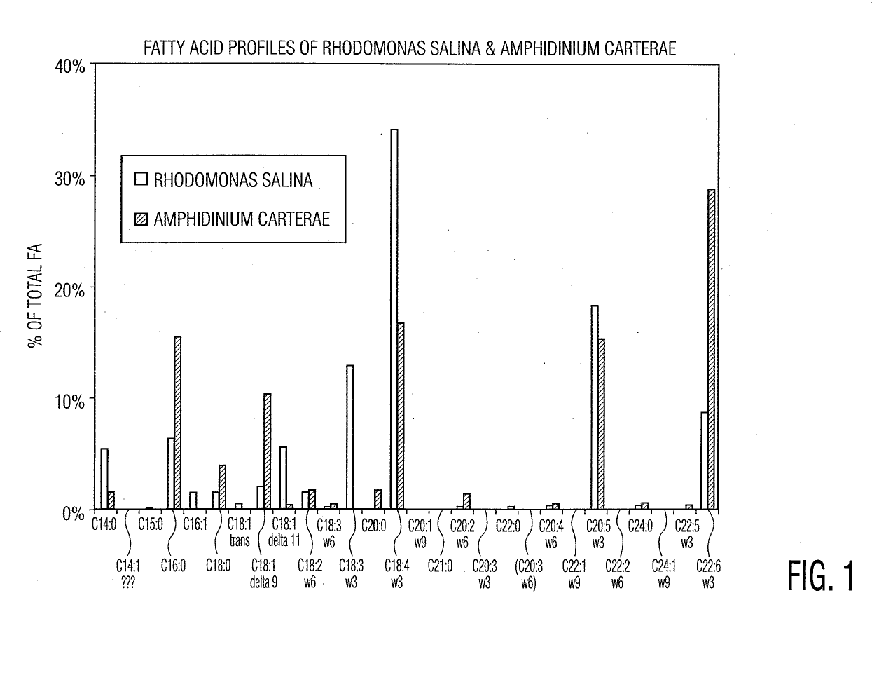

Rhodomonas salina and Amphidinium carterae were determined to contain significant amount of SDA (−34% and 17%, respectively) (FIG. 1). In addition, both species were found to produce EPA and DHA, which are the main components of fish oil. Alpha-linolenic acid (ALA), the immediate precursor of SDA, was quite high in R. salina, but not in A. carterae, indicating a low level of activity for A-6 desaturase, which converts ALA to SDA.

### Example 6

### Growth Characterization

Light intensity and temperature are two most important environmental factors that affect the growth of microalgae. To determine the optimal growth conditions for R. salina and A. carterae, their requirements for light intensity and temperature were defined.

A. Growth Characteristics of Rhodomonas salina.

Effects of Light Intensity on the Growth of R. salina.

Cells of R. salina were subjected to different light intensities, ranging from 20 to 200 μmol m⁻² s⁻¹ at room temperature. Samples were withdrawn daily and the growth of R. salina was measured as Chl-α and cell number.

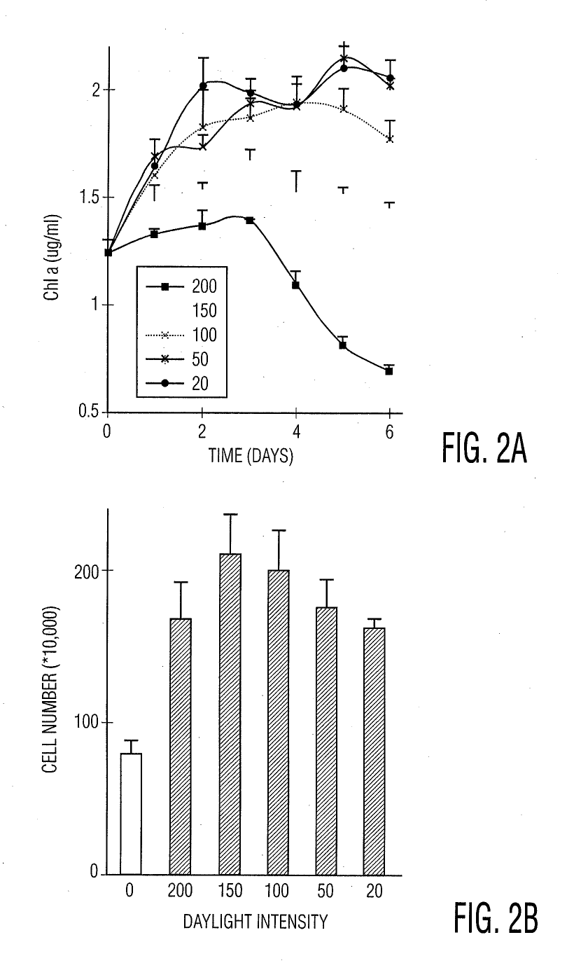

The optimal light intensity was below 100 μmol m⁻² s⁻¹ when growth was measured as an increase in Chl-α (FIG. 2A). A light intensity of 200 μmol m⁻² s⁻¹ caused a sharp decline after a moderate increase in the first three days. This result indicated that low light intensity was more favorable for R. salina, and high light intensity may cause photoinhibition leading to slower growth of R. salina. The same is true when cell number was used to assess the growth of R. salina. Thus, after one week, the highest cell concentration was obtained from the culture under a light intensity of 100 to 150 μmol m⁻² s⁻¹ (FIG. 2B). It should be noted that under these growth conditions, the final cell concentration reached over 2×10⁶ cells/ml, which is ten times higher than results obtained in our preliminary studies. This improvement may be due to the change of culture medium from ES-enriched seawater medium to f/2 medium.

Effects of Temperature on the Growth of R. salina.

Cells of R. salina were subjected to different temperatures which were controlled in a water bath at 14° C., 22° C., 28° C., and 34° C. under a light intensity of 50 μmol m⁻² s⁻¹. Samples were withdrawn daily and the growth of the R. salina was measured as Chl-α and cell number.

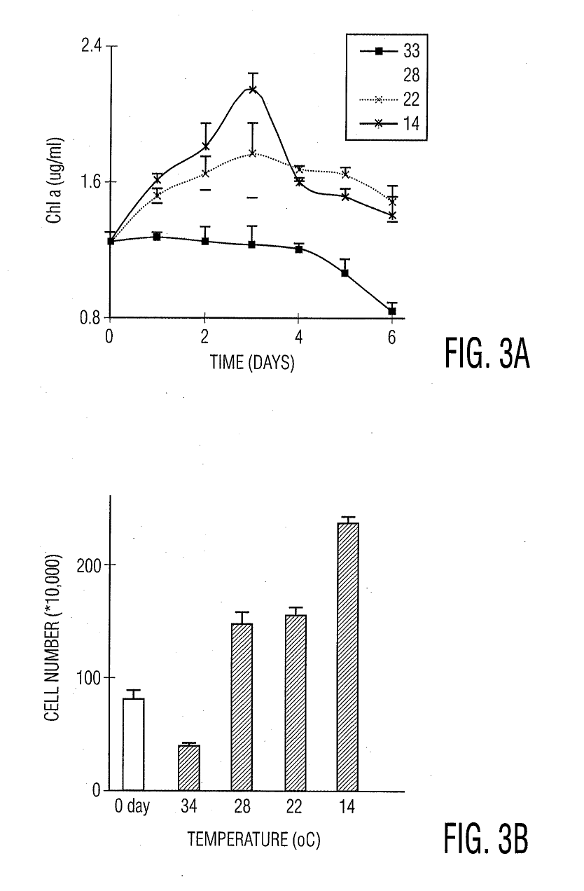

The optimal temperature for R. salina was found to be 14° C. when growth was measured as either an increase in Chl-α (FIG. 3A) or cell number (FIG. 3B). Growth wasslower at 22° C. and 28° C. when compared to that at 14° C. No growth was detectable at 34° C., and declines in both Chl-α and cell number were observed after three days at this temperature. As a marine species, R. salina cannot tolerate the high temperature of 34° C., even 28° C. caused a significant slow down in growth.

Effects of Light Intensity and Temperature on Total Pigments Profiles.

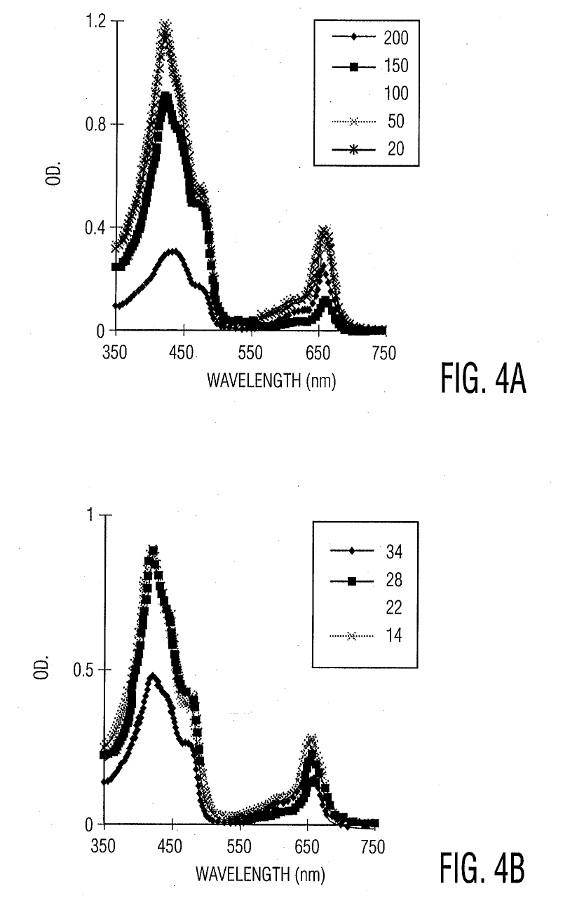

To further analyze the effects of light intensity and temperature on R. salina, cells grown under different light intensities and temperatures were harvested by filtration and total pigments were extracted with methanol. The pigment profiles are shown in FIG. 4. Two standard peaks of chlorophylls were observed at around 666 nm and 440 nm with a carotenoids shoulder at around 480 nm. Although the different light intensities and temperatures showed a clear impact on the absolute amounts of total pigments, the patterns of pigment profiles were not significantly different from each other, indicating that the light intensities and temperatures tested do not significantly affect the pigment profile.

B. Growth Characteristics of Amphidinium carterae.

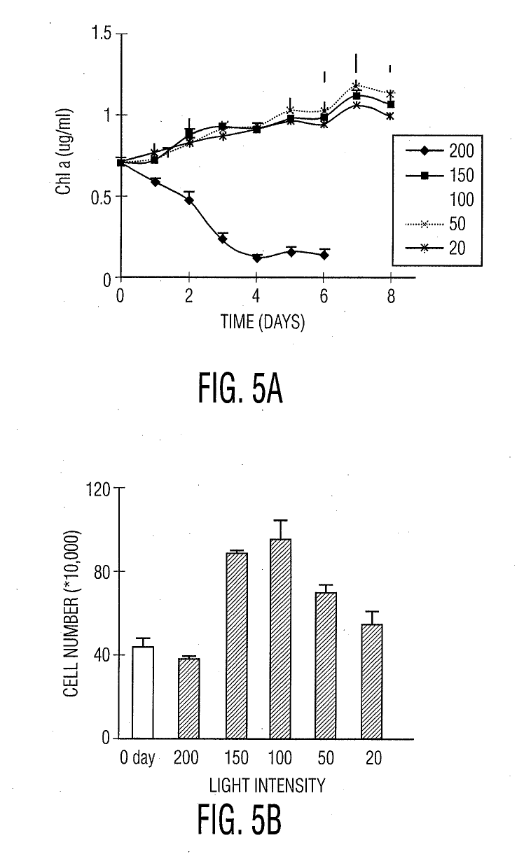

Effects of light intensity on A. carterae. Cells of A. carterae were subjected to different light intensities, ranging from 20 to 200 μmol m⁻² s⁻¹ at room temperature. Samples were withdrawn daily from the culture flasks and the growth of the A. carterae was measured as Chl-α and cell number. When growth was measured as an increase in Chl-α, the light intensities from 20 to 150 μmol m⁻² s⁻¹ had no significant effect on growth (FIG. 5A). In contrast, a light intensity of 200 μmol m⁻² s⁻¹ caused a rapid decline in Chl-α and eventually the bleaching of the culture. When the growth was measured as increase in cell number, the optimal light intensity was in the range of 100 to 150 μmol m⁻² s⁻¹. No growth was observed at a light intensity of 200 μmol m⁻² s⁻¹ (FIG. 5B). These results indicate that the microalgae A. carterae is very sensitive to high light intensity and can adapt to low light intensity for a reasonable growth rate.

Effects of Temperature on A. carterae.

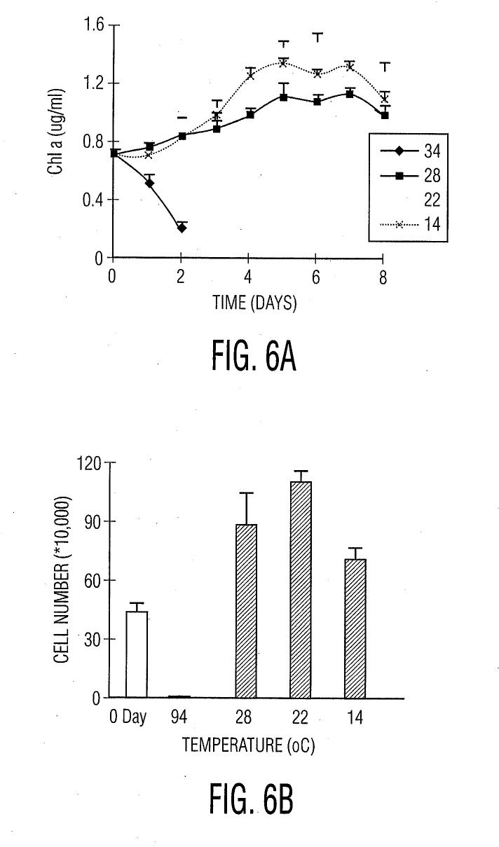

Cells of A. carterae were subjected to different temperatures controlled by a water bath at 14° C., 22° C., 28° C., 34° C. and under a light intensity of 50 μmol m⁻² s⁻¹. Samples from the cultures were withdrawn daily and the growth of the A. carterae was measured as Chl-α and cell number. The optimal temperature for A. carterae was found to be a temperature of 22° C. when growth was measured as an increase in Chl-α (FIG. 6A) and in cell number (FIG. 6B). At 14° C., the growth rate was similar to that at 22° C., but the final cell concentration was lower. No growth detected at 34° C.; instead a decline in both Chl-α and cell number was observed.

### C. Effects of Light Intensity and Temperature on Total Pigments Profiles

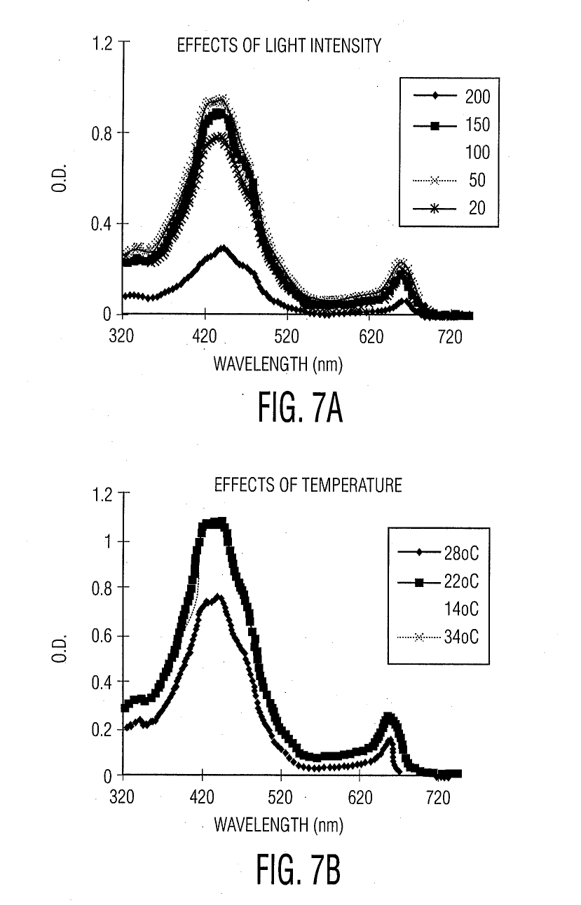

The pigment profiles of A. carterae are shown in FIG. 7. Similar to R. salina, the pigment profile pattern was not significantly different between the different treatments (light intensity and temperature); however, the absolute amount of total pigments was different under the different test conditions.

D. Cytotoxicity Tests for R. salina and A. carterae

Cytotoxicity of marine algae is a concern, especially when the algae are used for aquaculture feed or human nutrition. Therefore, R. salina and A. Carterae were tested to determine whether they were toxic or not. A brine shrimp cytotoxicity assay was employed for the test and another marine microalga, Navicular-like diatom (NLD), was used as negative control. Microalgal cells at various concentrations were distributed in wells of 96-well plates, newly hatched brime shrimp larvae (nauplii) were introduced to each well at a density around 10 nauplii per well. Wells containing medium only without microalgae served as a background control. The numbers of live nauplii were counted daily to monitor the survival rate.

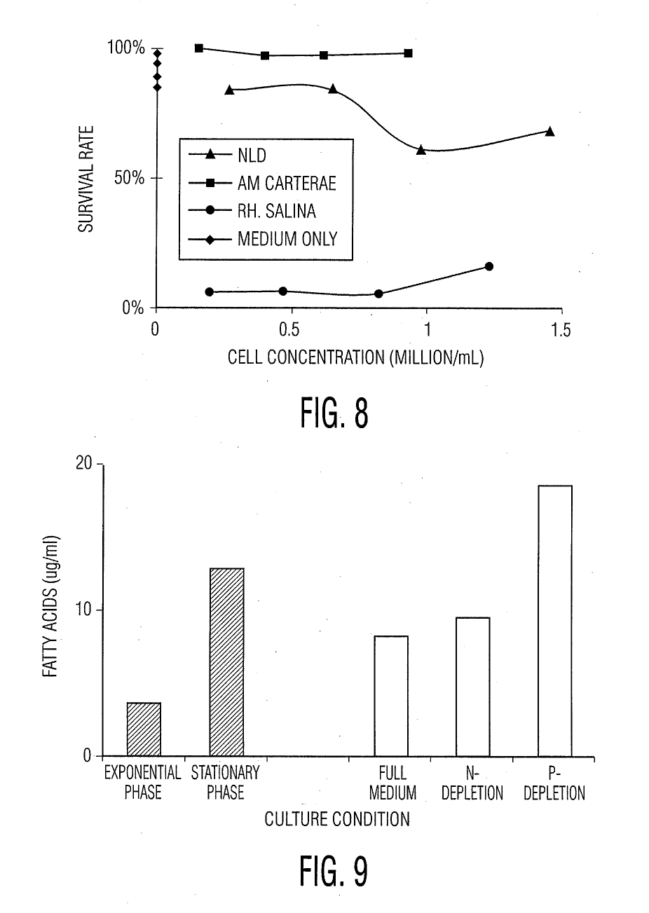

As shown in FIG. 8, R. salina did not show any adverse effect on the nauplii, which continued to grow for several days until cells of R. salina were depleted. The NDL showed a survival rate of 70%-90%, which was similar as the rate obtained from background (medium only no microalgae). For A. carterae, the results were quite surprising: after 24 hours more than 50% nauplii were dead; after 48 hours less than 10% survived. It is clear that A. carterae is toxic to brime shrimp nauplii. The mechanism of the toxicity was not determined. These results demonstrate that R. salina is not cytotoxic to brime shrimp, while A. carterae showed a clear toxicity.

### Example 7

### Determining the Conditions for Fatty Acids Accumulation

Based on results obtained from cytotoxicity tests, R. salina was chosen for further characterization on fatty acids accumulation and scale-up production. The following experiments were designed to test for methods that can increase the accumulation of fatty acids in R. salina.

Effect of Culture Stage of R. salina.

A typical batch culture of microalgae includes three stages: (1) a lag phase—the beginning of the culture, an adaptation period with low growth rate; (2) an exponential phase, the fastest growing period with rapid cell division; and (3) a stationary phase—due to nutrient depletion, the growth slows down accompanied by accumulation of secondary metabolites.

The following experiments were carried out in order to determine the growth stage during which R. salina accumulates large amounts of fatty acids. R. salina cells were inoculated into f/2 growth medium under the previously determined optimal light intensity and temperature (22° C. and 100 μmol m⁻² s⁻¹). Cells were harvested at exponential phase and stationary phase, respectively, for fatty acids analysis.

R. salina cells at stationary phase were determined to contain three times higher fatty acids levels than cells at exponential phase (FIG. 9; blue bars). This result is of interest for designing a production strategy for fatty acids from R. salina. For example, cells can be first cultivated under optimal conditions to obtain maximum biomass, which can be maintained in stationary phase to accumulate the desirable fatty acids prior to harvesting.

Effect of Nutrition Depletion

An effective method of inducing fatty acids accumulation in microalgae is to subject the cells to nutritional depletion, most commonly nitrogen or phosphorus starvation (Cohen, Z. and C. Ratledge, Single Cell Oils, American Oil Chemists' Society, Champaign, Ill., USA (2005)). To test the feasibility of this method, R. salina cells were washed three times with nitrogen-free or phosphorus-free f/2 medium, and then grown in the same medium for six days. Cells were harvested at the end of six days and fatty acid levels were measured (FIG. 9, right side, yellow bars).

As compared to the control, nitrogen starvation did not induce a significant accumulation of fatty acids. In contrast, phosphorus-free medium induced a significant increase in fatty acid content. This result suggests that for the mass production of SDA, phosphorus starvation can be employed to induce the accumulation of fatty acids.

Effect of Temperature.

Temperature is one the factors that can affect fatty acid accumulation in microalgae. Thus, in the following example, the effects of temperature on the accumulation of SDA in R. salina and A. carterae were studied.

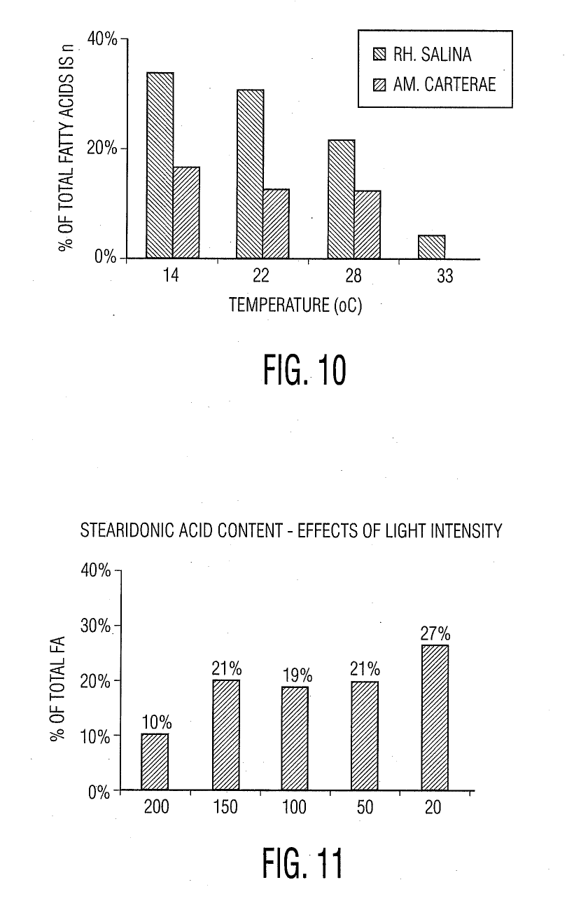

R. salina and A. carterae cells were inoculated in the full f/2 growth media under a low light intensity of 50 μmol m⁻² s⁻¹ and subjected to a temperature of 14° C., 22° C., 28° C., or 33° C. After one week of growth, cells were harvested by filtration and lipids were extracted and analyzed for fatty acid content. As shown in, FIG. 10, the SDA content of R. salina was determined to be significantly higher at the lower temperatures of 14° C. and 22° C. than at the higher temperature of 33° C. (FIG. 10). A similar trend was observed for A. carterae, which has less overall SDA content compared to R. salina. It is noted that the temperature of 14° C.

That which is claimed is:

1. A process of making a polyunsaturated fatty acid composition comprising at least 8% polyunsaturated fatty acids in weight, the process comprising:
extracting the polyunsaturated fatty acids from a microalgae, wherein,
(a) GLA is in an amount of 1% to 10% of total fatty acids;
(b) SDA is in an amount of 5% to 50% of total fatty acids;
(c) EPA is in an amount of 2% to 30% of total fatty acids; and
(d) DHA is in an amount of 2% to 30% of total fatty acids;
wherein the composition comprises at least 8% polyunsaturated fatty acids in weight.

2. The process of claim 1, wherein the microalgae is a microalgae having a cell wall of reduced thickness as compared to the wild-type microalgae, wherein said cell wall of reduced thickness improves extractability and/or bioavailability of the microalgal lipid fraction.

3. The process of claim 1, wherein the microalgae is selected from the group consisting of Dinophyceae, Cryptophyceae, Trebouxiophyceae, Pinguiophyceae, and combinations thereof.

4. The process of claim 1, wherein said microalgae is selected from the group consisting of Parietochloris spp., Rhodomonas spp., Porphyridium spp., and combinations thereof.

5. The process of claim 1, wherein said microalgae is Rhodomonas salina.

6. A process of making a composition comprising at least 5% stearidonic acid, the process comprising:
(a) cultivating a microalgae to produce a microalgal biomass; and either
(b) extracting said microalgal oil from said cultivated microalgae; or
(c) removing water from said microalgal biomass to achieve a solids content from 5 to 100% weight percent;
wherein the composition comprises at least 5% stearidonic acid.

7. A process of making an animal feed additive comprising fatty acids from a microalgae, the process comprising:
(a) cultivating microalgae to produce a microalgal biomass; and either
(b) extracting microalgae oil from said microalgal biomass to produce a microalgal oil; or
(c) removing water from said microalgal biomass to produce a microalgal biomass with a solids content from 5% to 100% weight percent;
wherein the animal feed additive comprises fatty acids from a microalgae.

8. The process of claim 7 wherein the fatty acids are short chain omega-3 fatty acids.

9. A process of making an animal feed additive comprising at least 8% polyunsaturated fatty acids in weight; the process comprising:
extracting the fatty acids from a microalgae, wherein,
(a) GLA is in an amount of 1% to 10% of total fatty acids;
(b) SDA is in an amount of 5% to 50% of total fatty acids;
(c) EPA is in an amount of 2% to 30% of total fatty acids: and
(d) DHA is in an amount of 2% to 30% of total fatty acids:
wherein the animal feed additive comprises at least 8% polyunsaturated fatty acids.

10. A process of producing an animal having an increased tissue content of long chain omega-3 fatty acids, the method comprising feeding to an animal an animal feed additive comprising microalgal polyunsaturated fatty acids, the microalgal polyunsaturated fatty acids comprising: (a) a microalgal oil extracted from a cultivated microalgae biomass and/or (b) a microalgal biomass from a cultivated microalgae, wherein water is removed from microalgal biomass to achieve a solids content from 5 to 100%; wherein an animal is produced having increased tissue content of long chain omega-3 fatty acids.

11. A process of producing an animal having an increased tissue content of long chain omega-3 fatty acids, the process comprising feeding to an animal an animal feed additive comprising at least 8% polyunsaturated fatly acids in weight; the animal feed additive comprising polyunsaturated fatty acids extracted from a microalgae, wherein (a) GLA is in an amount of 1% to 10% of total fatty acids; (b) SDA is in an amount of 5% to 50% of total fatty acids; (c) EPA is in an amount of 2% to 30% of total fatty acids; and (d) DHA is in an amount of 2% to 30% of total fatty acids; wherein an animal is produced having an increased tissue content of long chain omega-3 fatty acids.

12. The process of claim 10, wherein the animal is selected from the group consisting of fish, poultry, pigs, sheep, and beef and dairy cattle.

13. The process of claim 11, wherein the animal is selected from the group consisting of fish, poultry, pigs, sheep, and beef and dairy cattle.

14. A method of treating a mammalian disease in a subject in need thereof by administration to the subject a therapeutically effective amount of a polyunsaturated fatty acid composition comprising at least 8% polyunsaturated fatty acids in weight extracted from a microalgae, wherein the microalgae fatty acid extract comprises,
(a) GLA in an amount of 1% to 10% of total fatty acids;
(b) SDA in an amount of 5% to 50% of total fatty acids;
(c) EPA in an amount of 2% to 30% of total fatty acids, and
(d) DHA in an amount of 2% to 30% of total fatty acids.

15. A method of treating a mammalian disease in a subject in need thereof by administration to the subject a therapeutically effective amount of a composition comprising at least 5% SDA in a non-phospholipid form, the composition comprising either (a) a microalgal oil extracted from a cultivated microalgae biomass or (b) a microalgal biomass from a cultivated microalgae, wherein water is removed from microalgal biomass to achieve a solids content from about 5 to 100% weight percent.

16. A polyunsaturated fatty acid composition made by the process of claim 1.

17. The composition of claim 16, wherein the GLA further comprises GLA from borage oil.

18. A polyunsaturated fatty acid composition made by the process of claim 6.

19. The composition of claim 18, wherein the GLA further comprises GLA from borage oil.

20. A food product comprising: (a) from 0.01-99.99 percent by weight of the composition of claim 16 in combination with (b) from 99.99 to 0.01 percent by weight of at least one additional ingredient selected from the group consisting of proteins, carbohydrates and fiber, and combinations thereof.

21. A food product comprising: (a) from 0.01-99.99 percent by weight of the composition of claim 18 in combination with (b) from 99.99 to 0.01 percent by weight of at least one additional ingredient selected from the group consisting of proteins, carbohydrates and fiber, and combinations thereof.

22. The food product of claim 20, wherein the food product is in the form of a cereal, an extruded bar, a food topping, an ice cream, a drink, a candy, a snack mix, or a baked product.

23. The food product of claim 21, wherein the food product is in the form of a cereal, an extruded bar, a food topping, an ice cream, a drink, a candy, a snack mix, or a baked product.

24. An animal feed additive made by the process of claim 7.

25. An animal feed additive made by the process of claim 9.

26. An animal product produced by feeding to an animal the animal feed additive of claim 24.

27. An animal product produced by feeding to an animal the animal feed additive of claim 25.

28. The animal product of claim 26 comprising eggs, milk, or meat.

29. The animal product of claim 27 comprising eggs, milk, or meat

30. An animal produced by the method of claim 10.

31. An animal produced by the method of claim 11.

32. An animal product produced from the animal of claim 30.

33. An animal product produced from the animal of claim 31.
